(19) United States
(12) Patent Application Publication
(10) Pub. No.: US 2003/0175925 A1
(43) Pub. Date: Sep. 18, 2003

(54) Nicotinamide ribonucleoside kinase

(76) Inventors: Oleg Kurnasov, Chicago, IL (US); Boris Polanuyer, Skokie, IL (US); Yakov Kogan, Chicago, IL (US); Andrei Osterman, Chicago, IL (US)

Correspondence Address: STERNE, KESSLER, GOLDSTEIN & FOX PLLC, 1100 NEW YORK AVENUE, N.W., WASHINGTON, DC, 20005, US

(21) Appl. No.: 09/793,705

(22) Filed: Feb. 27, 2001

(51) Int. Cl. C12N 9/12

(52) U.S. Cl. 435/194

(57) ABSTRACT

The present invention relates to novel nicotinamide ribonucleoside kinase nucleic acids, polypeptides, and uses thereof.

### BACKGROUND OF THE INVENTION

1. Field of the Invention

The present invention relates to novel nicotinamide ribonucleoside kinase nucleic acids, polypeptides, and uses thereof.

2. Background Art

Nicotinamide dinucleotide (NAD⁺ and NADH) and its phosphorylated analogs are indispensable co-factors for numerous oxydoreductases in all living organisms. Additionally, NAD⁺ is a substrate for a variety of enzymes, such as bacterial DNA ligase, and various ADP-ribosylating factors both in prokaryotes and eukaryotes. In contrast to red-ox processes, the latter reactions deplete the NAD pool. In E. coli, the half-life of the NAD pool is about 90 minutes under aerobic growth conditions. Most lining organisms are thought to be able to recycle NAD, although not all organisms are able to synthesize NAD⁺ de novo, or to scavenge niacin from the growth medium.

Enzymatic steps involved in NAD/NADP biosynthesis, recycling and salvage as studied in E. coli and Salmonella, are shown in FIG. 1A. Some of the corresponding genes, experimentally identified mostly in E. coli and Salmonella, as well as their orthologs in a number of sequenced bacterial genomes, are listed in Table 1A. However, for a number of enzymes shown in FIG. 1A, including Nicotinamide Ribonucleoside Kinase (NRKse), corresponding genes have not been previously identified.

Nicotinamide ribonucleoside kinase (NRKse) (EC 2.7.1.22) is an enzyme that is involved in the NAD salvage and recycling pathways. Specifically, NRKse phosphorylates β-nicotinamide ribonucleoside (NmR) to produce β-nicotinamide mononucleotide (NMN), as shown in FIGS. 1A. H influenzae and other members of Pasteurellaceae family lack most of NAD biosynthetic genes present in E. coli and other Enterobacteriaceae (see Table 1A), which results in significant differences in NAD metabolism between these organisms.

Pasteurellaceae can not synthesize NAD de novo, but some of them (V-factor independent species. such as H. ducrei) can use niacin as a biosynthetic precursor of NAD, due to the presence of Nicotinamide Phosphoribosyl Transferase (nadV). H. influenzae lacks nadV (see Table 1A) and it must use NRKse to metabolize one of the established V-factors, NmR, and NRKse is thought to be an essential enzyme in H. influenzae. Likewise, NRKse is thought to be an essential enzyme in other V-factor dependent Haemophilus species, and other members of the Pasteurellaceae family.

Although NRKse activity has previously been detected in microbes such as Salmonella and E. coli, the polynucleotide sequence encoding NRKse was not identified prior to the research described herein. Thus, the NRKse gene was considered to be a “missing” gene.

### BRIEF SUMMARY OF THE INVENTION

The invention provides isolated nucleic acid molecules encoding NRKse.

In E. coli and in several other bacteria, NRKse is encoded by the C-terminal domain of NadR. The boundaries of the NRKse domain (P-loop kinase) in each NadR protein are indicated in Table 1B. Thus, the present invention features novel, isolated NRKse polynucleotides and polypeptides, vectors, host cells, antibodies, and recombinant methods for producing the polypeptides and polynucleotides, as well as methods for using such polypeptides and polynucleotides.

More particularly, the invention features an isolated NRKse nucleic acid molecule comprising an isolated polynucleotide having a nucleotide sequence at least 95% identical to a sequence selected from the group consisting of:

(a) a nucleotide sequence encoding a biologically active polypeptide fragment of a nicotinamide ribonucleoside kinase (NRKse) domain of SEQ ID NO: 2, 4, 6, 8, 10, 12, 14, 16, 18, 20, 22, 24, 26, 28, 30, 32, 34, 36, 38, 40, or 42;

(b) a nucleotide sequence encoding an NRKse domain of SEQ ID NO: 2, 4, 6, 8, 10, 12, 14, 16, 18, 20, 22, 24, 26, 28, 30, 32, 34, 36, 38, 40, or 42; and

(c) a nucleotide sequence capable of hybridizing under stringent conditions to any one of the polynucleotides specified in (a) or (b).

In a preferred embodiment, the polynucleotide comprises a nucleotide sequence encoding a nicotinamide ribonucleoside kinase (NRKse). For example, the isolated polynucleotide preferably comprises a nucleotide sequence encoding the amino acid sequence identified as an NRKse domain of SEQ ID NO: 2, 4, 6, 8, 10, 12, 14, 16, 18, 20, 22,24, 26, 28, 30, 32, 34, 36, 38, 40, or 42.

In another preferred embodiment, the polynucleotide comprises the entire nucleotide sequence of an NRKse domain of SEQ ID NO: 1, 3, 5, 7, 9, 11, 13, 15, 17, 19, 21, 23, 25, 27, 29, 31, 33, 35, 37, 39, or 41. Alternatively, the nucleotide sequence can comprise sequential nucleotide deletions from either terminus of the nucleotide sequence encoding NRKse.

The invention also includes recombinant vectors comprising an isolated nucleic acid molecule as described above. Additionally, the invention includes a method of making a recombinant host cell comprising an isolated nucleic acid molecule described herein; the method comprises introducing the isolated nucleic acid molecule into a host cell, thereby producing a recombinant host cell.

Such recombinant host cells, which may also comprise vector sequences, are included within the invention.

The invention also features an isolated NRKse polypeptide comprising an amino acid sequence at least 95% identical to a sequence selected from the group consisting of:

(a) a biologically active polypeptide fragment of an NRKse domain of SEQ ID NO: 2, 4, 6, 8, 10, 12, 14, 16, 18, 20, 22, 24, 26, 28, 30, 32, 34, 36, 38, 40, or 42 and

(b) a polypeptide comprising the sequence of an NRKse domain of SEQ ID NO: 2, 4, 6, 8, 10, 12, 14, 16, 18, 20, 22, 24, 26, 28, 30, 32, 34, 36, 38, 40, or 42.

In a preferred embodiment, the polypeptide comprises sequential amino acid deletions from either the C-terminus or the N-terminus of NRKse.

Also included within the invention is an isolated antibody that binds specifically to an isolated NRKse polypeptide described above. Preferably, the antibody preferentially binds to NRKse rather than to NadR. More preferably, the antibody binds to NRKse and does not bind to NadR.

In a related aspect, the invention includes a recombinant host cell that expresses the isolated NRKse polypeptide described above. The recombinant host cell be used in a method of making an isolated NRKse polypeptide by (a) culturing the recombinant host cell under conditions such that the NRKse polypeptide is expressed; and (b) recovering the NRKse polypeptide. The isolated polypeptide produced by such a method also is included within the invention.

In another aspect, the invention features a method for determining whether a compound is a modulator (e.g., an agonist or antagonist) of NRKse; the method comprises:(a) contacting a biologically active NRKse polypeptide with the compound; and (b) detecting an alteration in the activity of the polypeptide in the presence of the compound as an indication that the compound is a modulator of NRKse. Modulators identified by such a method can be used to modulate NRKse activity in an organism, by contacting the organism with the modulator. For example, such modulators can be used as antiinfectives, for example, when the organism is a pathogen and modulation comprises decreasing NRKse activity in the organism, thereby inhibiting the pathogenicity of the organism. Alternatively, the modulator can be used to increase NRKse activity in the organism. For example, NRKse activity can be increased in bacterial strains used in the production of bulk chemicals, such as amino acids, under conditions in which the pool of NAD/NADP may otherwise limiting.

If desired, the aforementioned assay of NRKse activity may also include (c) contacting the compound with nicotinamide mononucleotide adenylyl transferase (NMNATse); and (d) detecting an alteration in the activity of the NMNATse in the presence of the compound as an indication that the compound is a modulator of NMNATse.

In another aspect, the invention features a method for producing β-nicotinamide mononucleotide (NMN) or an analog thereof, the method comprises contacting β-nicotinamide ribonucleoside (NMR) or an analog thereof with an isolated biologically active NRKse polypeptide of the invention, thereby producing NMN or an analog thereof. For example, the analog may be 3′deazaguanosine or tiazofurin. If desired, NMN or the analog thereof can then be contacted with nicotinamide mononucleotide adenylyl transferase (NMNATse), thereby producing β-nicotinamide adenine dinucleotide (NAD) or a dinucleotide of the analog. The biologically active NRKse polypeptide and NMNATse can be provided as a fusion protein, or provided as separate molecules.

### BRIEF DESCRIPTION OF THE FIGURES

FIG. 1A is a schematic representation of the NAD/NADP biosynthetic, salvage, and recycling pathways in bacteria such as E coli and Salmonella. The corresponding gene for NmR kinase (NRKse) previously was “missing.” FIG. 1B is a schematic representation of the NAD/NADP salvage and recycling pathways in Haemophilus influenzae.

FIG. 2 is a listing of the nucleotide sequences of polynucleotides encoding NadR proteins containing an NRKse domain in Salmonella typhimurium (SEQ ID NO: 1), Salmonella typhi (SEQ ID NO: 3), Escherichia coli (SEQ ID NO: 5), Yersinia pestis (SEQ ID NO: 7), Salmonella enteritidis (SEQ ID NO: 9), Klebsiella pneumoniae (SEQ ID NO: 11), Actinobacillus actinomycetemcomitans (SEQ ID NO: 13). Pasteurella multocida (SEQ ID NO: 15), Yersinia pseudotuberculosis (SEQ ID NO: 17), Klebsiella pneumoniae (SEQ ID NO: 19), Haemophilus influenzae (SEQ ID NO: 21), Haemophilus ducreyi (SEQ ID NO: 23), Salmonella enteritidis (SEQ ID NO: 25), Lactococcus lactis (SEQ ID NO: 27), Mycobacterium tuberculosis (SEQ ID NO: 29), Mycobacterium bovis (SEQ ID NO: 31), Nostoc punctiforme (SEQ ID NO: 33), Haemophilus ducreyi (SEQ ID NO: 35), Pseudomonas aeruginosa (SEQ ID NO: 37), Pseudomonas fluorescens (SEQ ID NO: 39), and Moroxella catarrhalis (SEQ ID NO: 41). The boundaries of the NRKse domain (P-loop kinase) are indicated in Table 2.

FIG. 3 is a listing of the amino acid sequence of NadR proteins containing an NRKse domain in Salmonella typhimurium (SEQ ID NO: 2), Salmonella typhi (SEQ ID NO: 4), Escherichia coli (SEQ ID NO: 6), Yersinia pestis (SEQ ID NO: 8), Salmonella enteritidis (SEQ ID NO: 10), Klebsiella pneumoniae (SEQ ID NO: 12), Actinobacillus actinomycetemcomitans (SEQ ID NO: 14), Pasteurella multocida (SEQ ID NO: 16), Yersinia pseudotuberculosis (SEQ ID NO: 18), Klebsiella pneumoniae (SEQ ID NO: 20), Haemophilus influenzae (SEQ ID NO: 22), Haemophilus ducreyi (SEQ ID NO: 24), Salmonella enteritidis (SEQ ID NO: 26), Lactococcus lactis (SEQ ID NO: 28), Mycobacterium tuberculosis (SEQ ID NO: 30), Mycobacterium bovis (SEQ ID NO: 32), Nostoc punctiforme (SEQ ID NO: 34), Haemophilus ducreyi (SEQ ID NO: 36), Pseudomonas aeruginosa (SEQ ID NO: 38), Pseudomonas fluorescens (SEQ ID NO: 40) and Moroxella catarrhalis (SEQ ID NO: 42).

FIG. 4 is an alignment of the amino acid sequences of the NadR proteins disclosed herein. The helix-turn-helix (HTH) motif, for DNA binding, is shown (e.g., amino acids 1-57 in RSY00616). The NTP binding motif, for NMNATse, is shown (e.g., amino acids 58-231 in RSY00616). The Walker A and Walker B motifs, for NmR kinase, are shown (amino acids 232-410 in RSY00616).

FIGS. 5A-5D are listings of the nucleotide and amino acid sequences of portions of genetic constructs used in the assays described herein. The His-Tag and TEV-protease sites are underscored. The first Met of the NMNATse sequence is in boldface print and underscored. FIG. 5A is a sequence of an essential portion of plasmid “pONadR_HI_N” containing PCR fragment “NadR_HI_Ndomain” amplified from the genomic DNA of H. influenzae using primers 43 and 44, listed in Table 3, and cloned to the expression vector using NcoI and SalI restriction enzymes both in the vector and in the fragment. This fragment encodes the N-terminal domain of NadR from H. influenzae (amino acids 38-212, only NMNATse domain). The second Ser-39 is replaced with Ala, and the stop codon is shown. FIG. 5B is a sequence from plasmid “pONadR_HI_T” containing PCR fragment “NadR_HI_Truncated” amplified from the genomic DNA of H. influenzae using primers 43 and 45, listed in Table 3, and cloned to the expression vector using NcoI and SalI restriction enzymes both in the vector and in the fragment. This fragment encodes the N-truncated of NadR protein from H. influenzae (amino acids 38-407, both NMNATse and NRKse domains). FIG. 4C is a sequence from plasmid “pONadR_SY” containing PCR fragment “NadR_SY” amplified from the genomic DNA of Salmonella typhimurium using primers 46 and 17, listed in Table 3, and cloned to the expression vector (pPROEX-HTa) using BspHI/NcoI and SalI restriction enzymes. This fragment encodes the full-size of NadR protein from Salmonella typhimurium (containing all three domains: HTH, NMNATse and NRKse). FIG. 5D is a listing of the sequence of the human NMNATse that was used in the coupled assay described herein. A sequence, from plasmid pONadD_HS, containing the NMNATse (NadD2_HS) coding region is shown.

FIG. 6 is a chromatogram illustrating the conversion of NmR to NMN and ADP by the NRKse contained within NadR in the presence of ATP (line A). In the absence of the NRKse, NmR is not converted to NMN (line B).

FIG. 7 is a schematic representation of a coupled assay in which NmR, in the presence of ATP, is converted by NRKse (i.e., NmR kinase) to NMN and ADP. In the presence of ATP, NMN then is converted by NMNATse to NAD³⁰ . In the presence of ethanol, NAD⁺ is converted by alcohol dehydrogenase (ADH) to NADH.

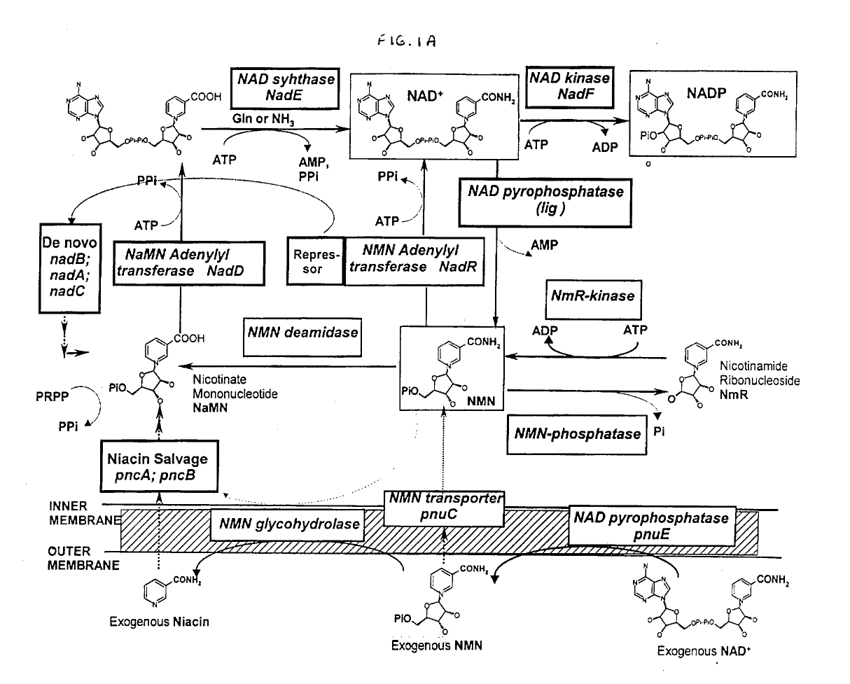

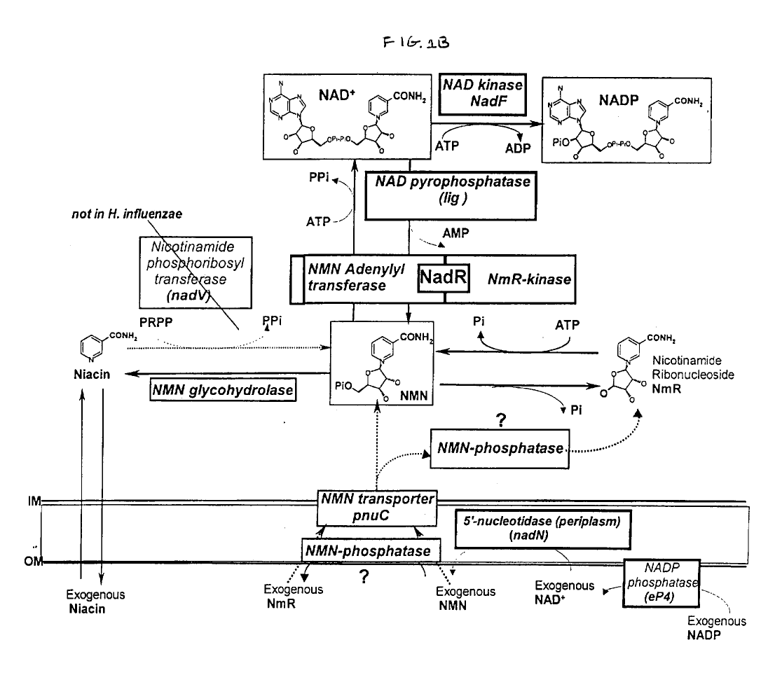

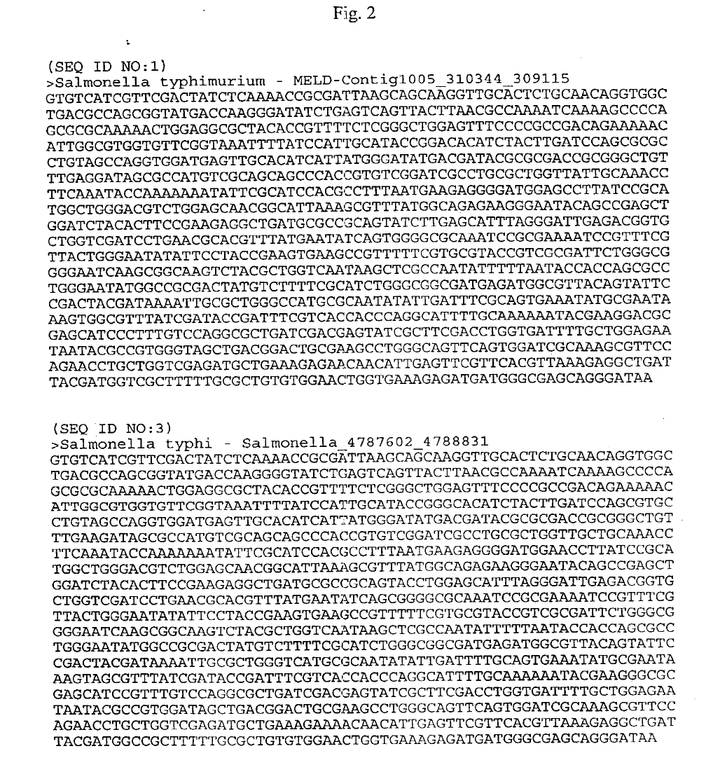

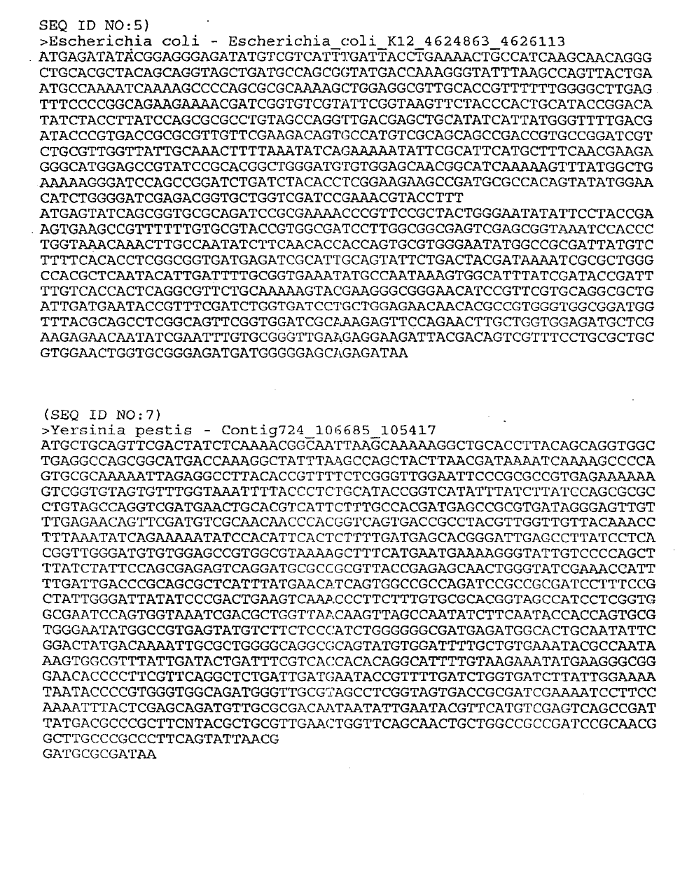

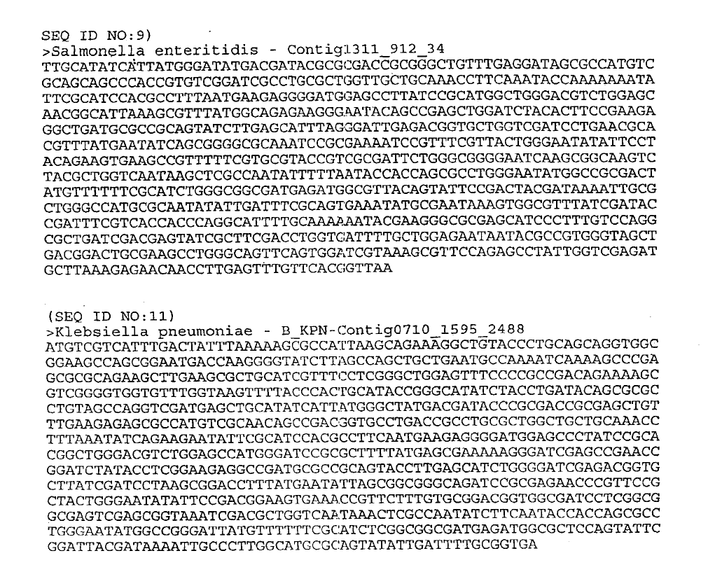

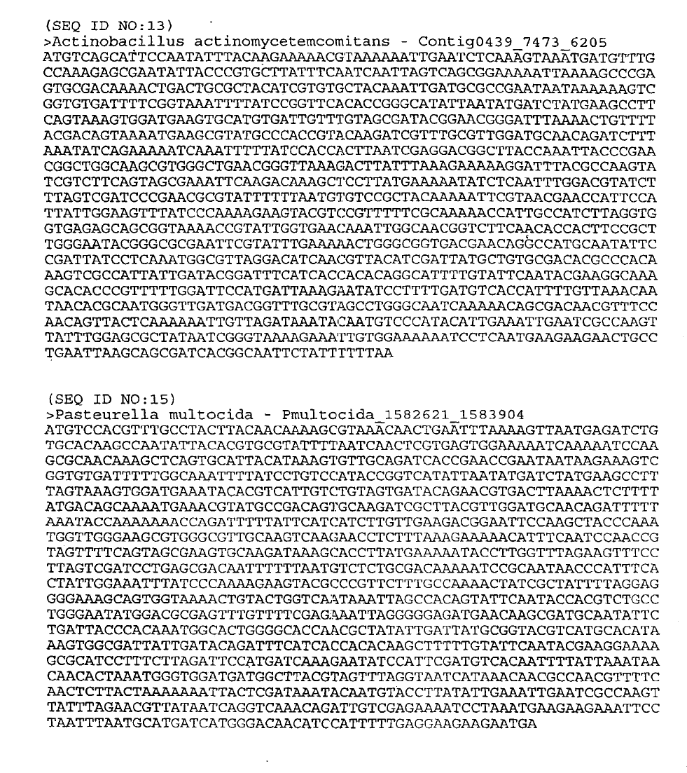

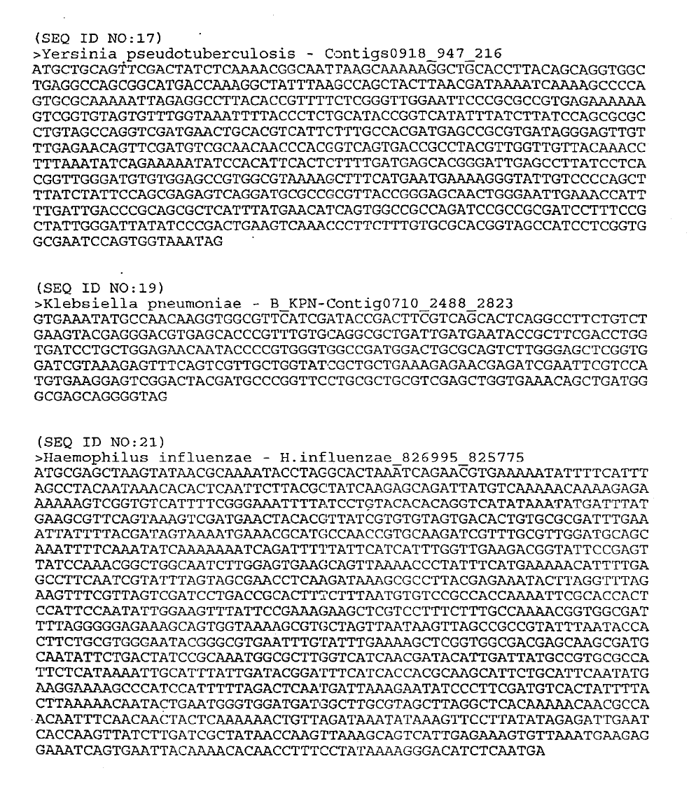

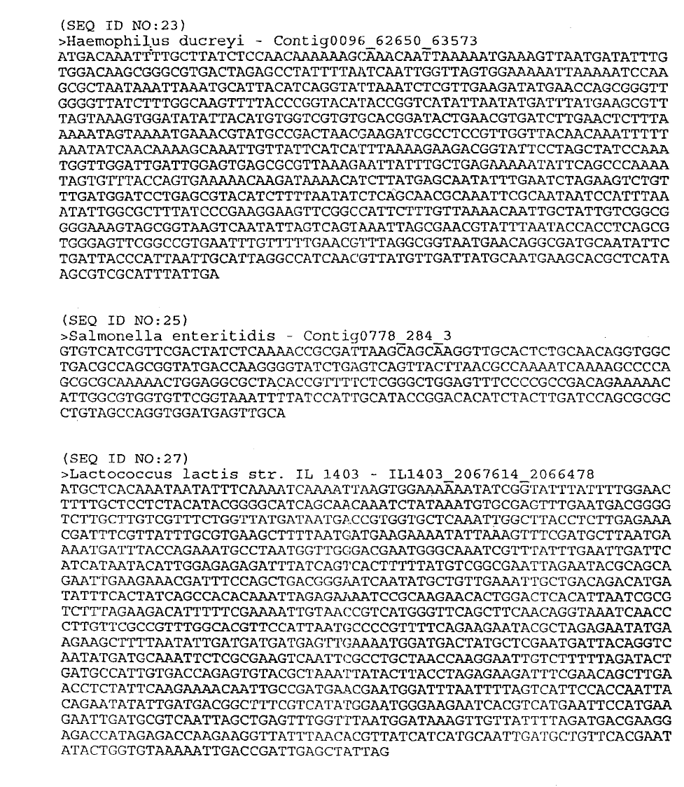

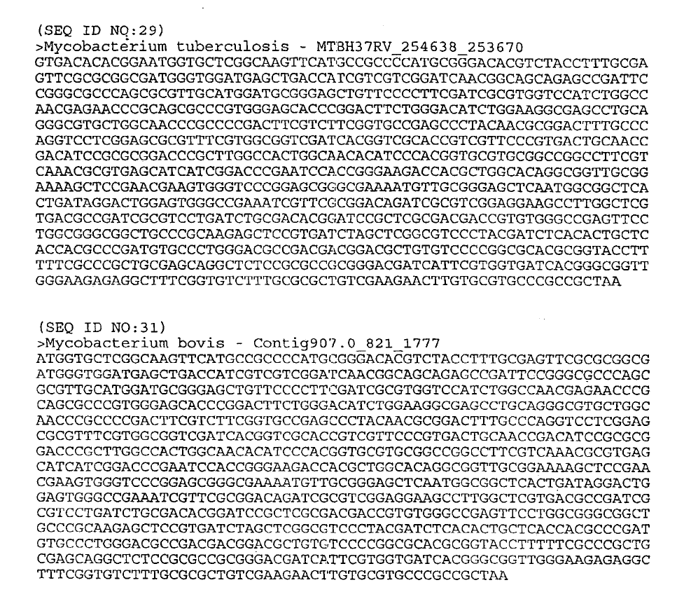

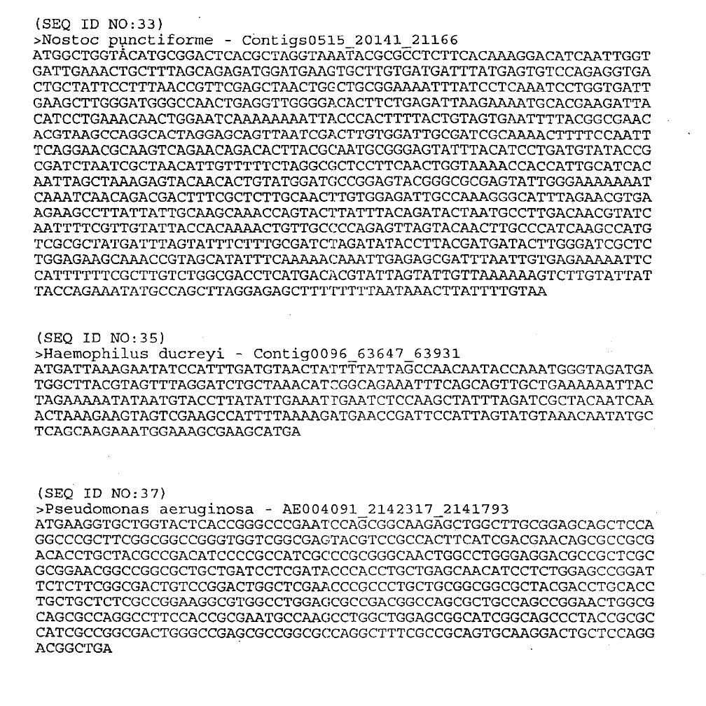

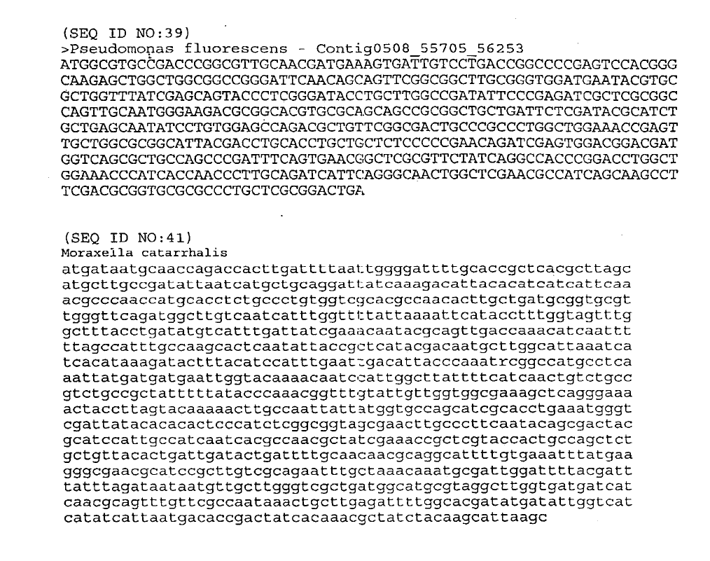

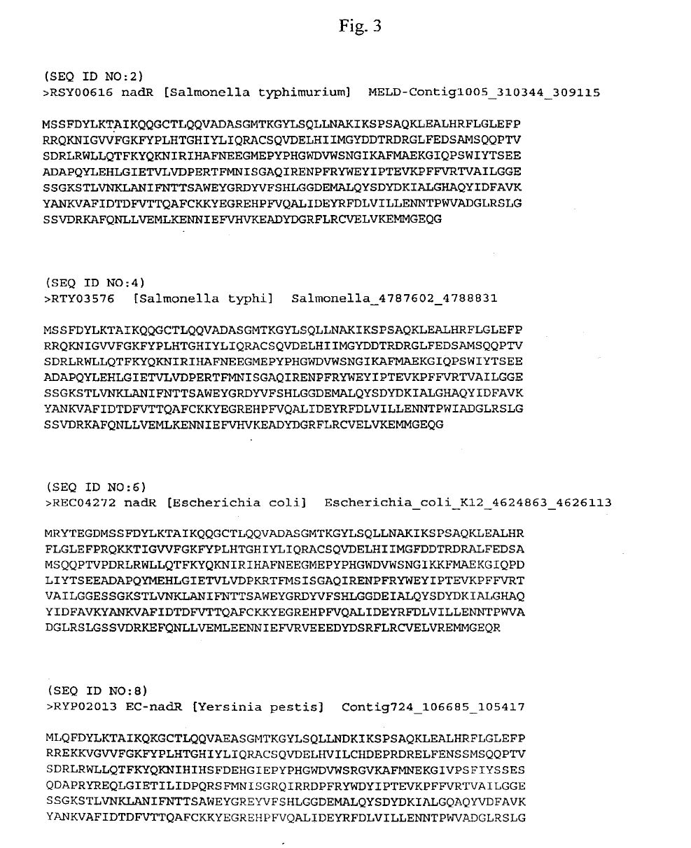

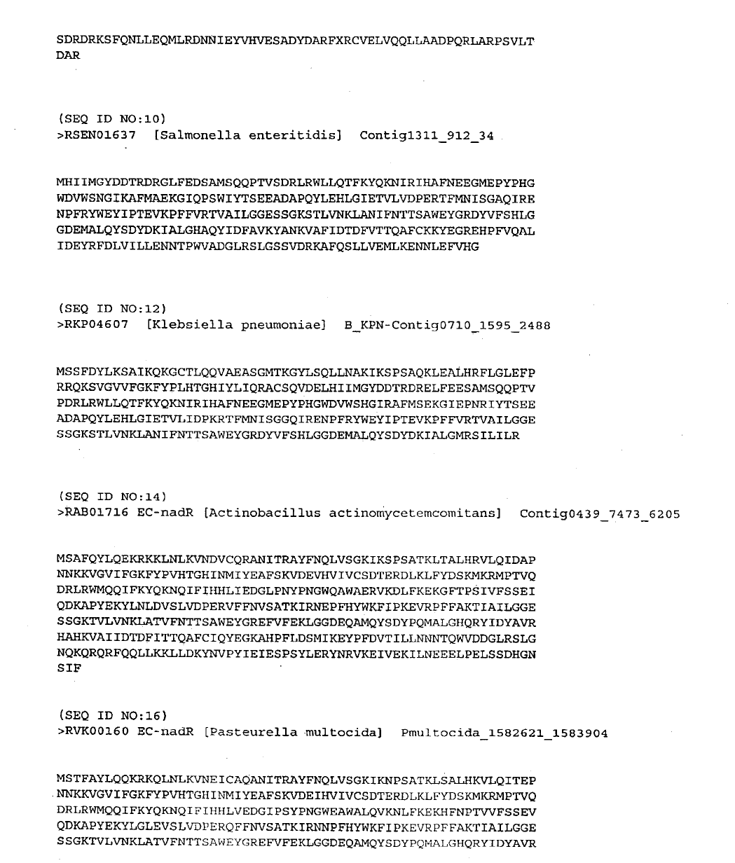

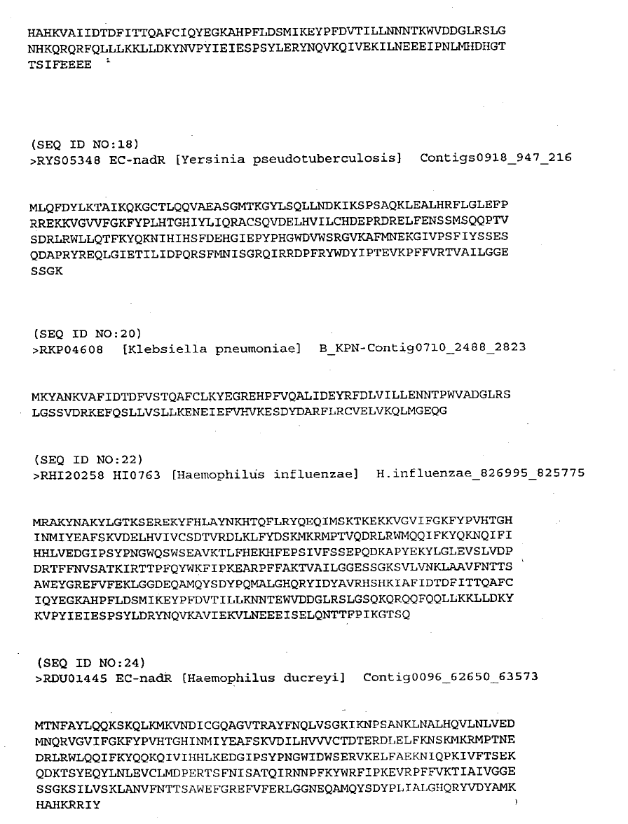

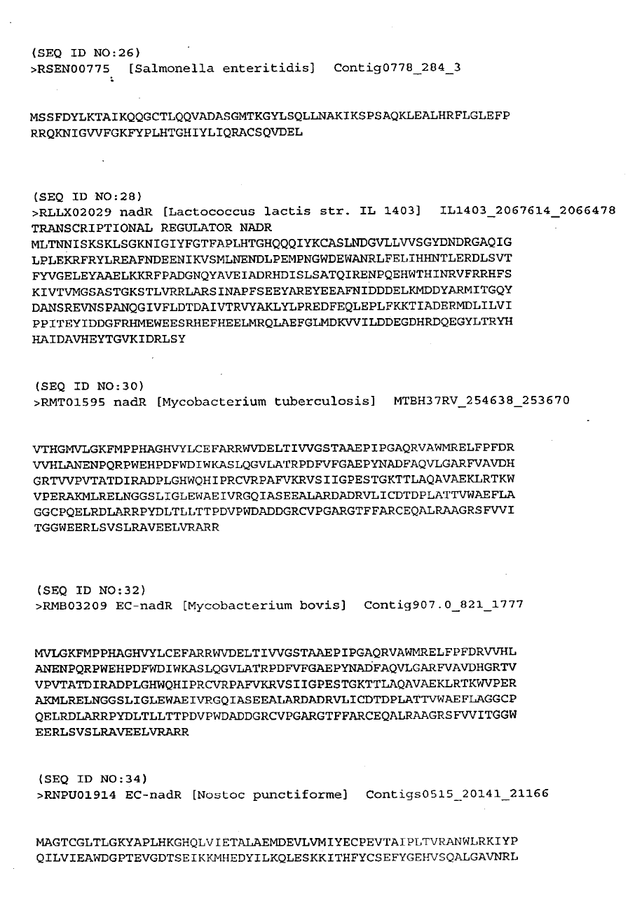

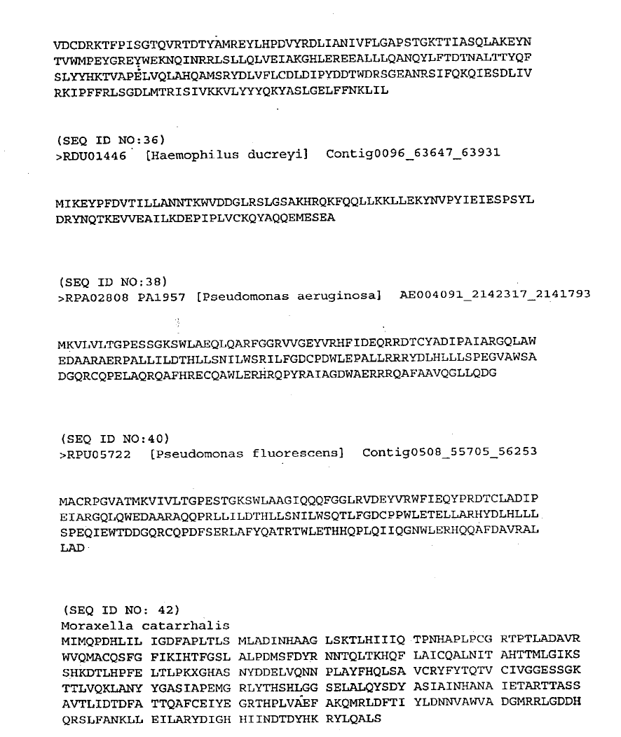

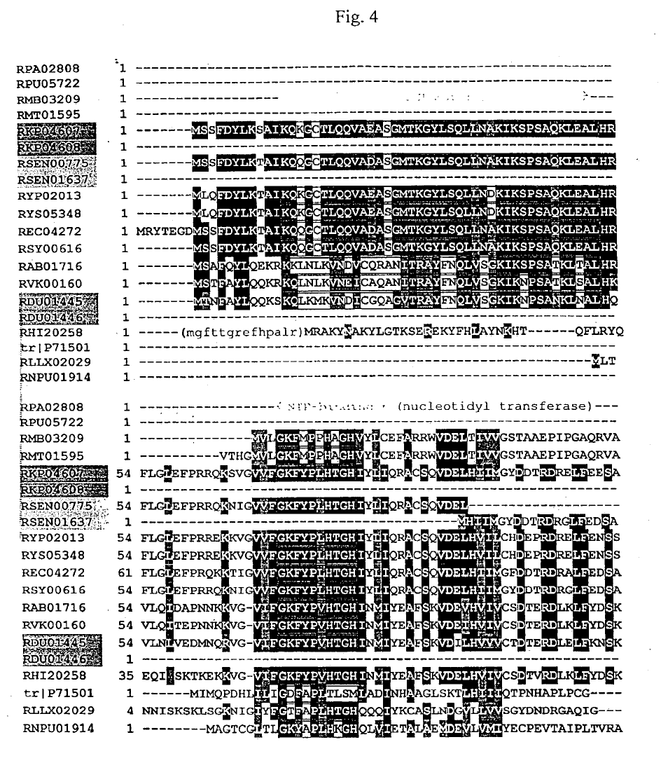

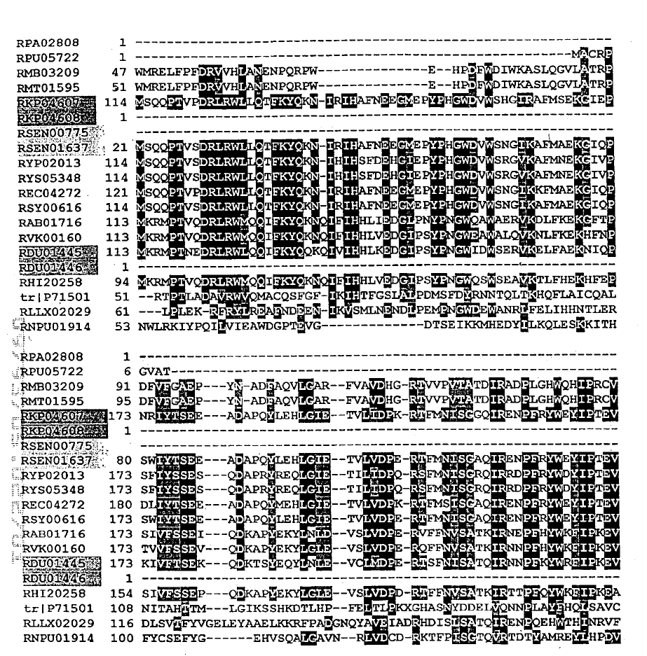

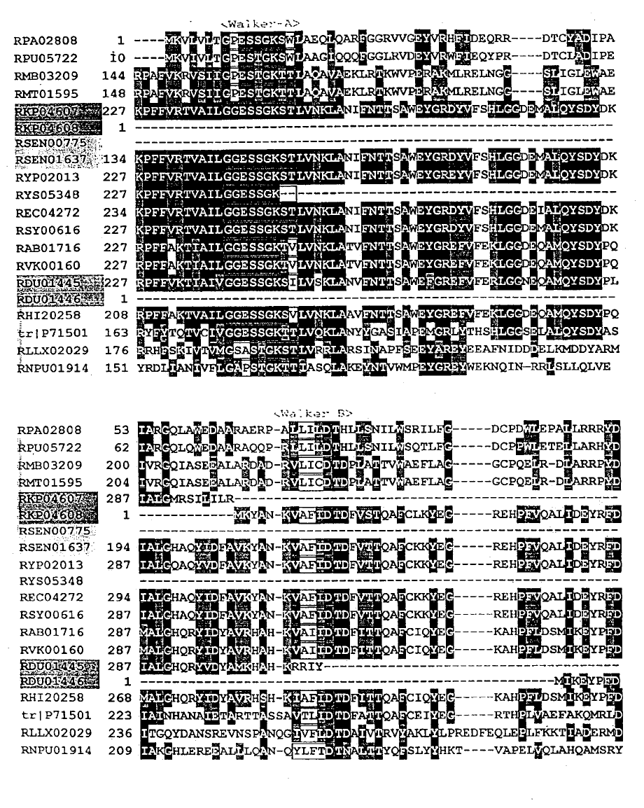

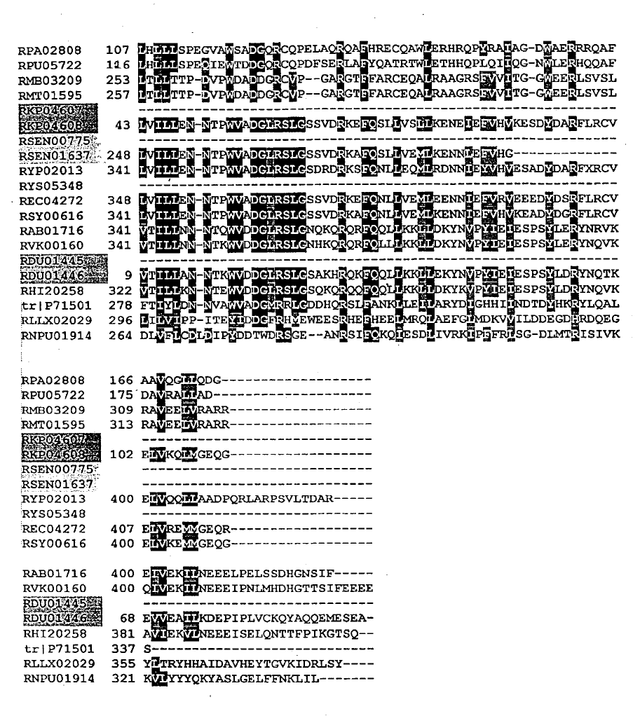

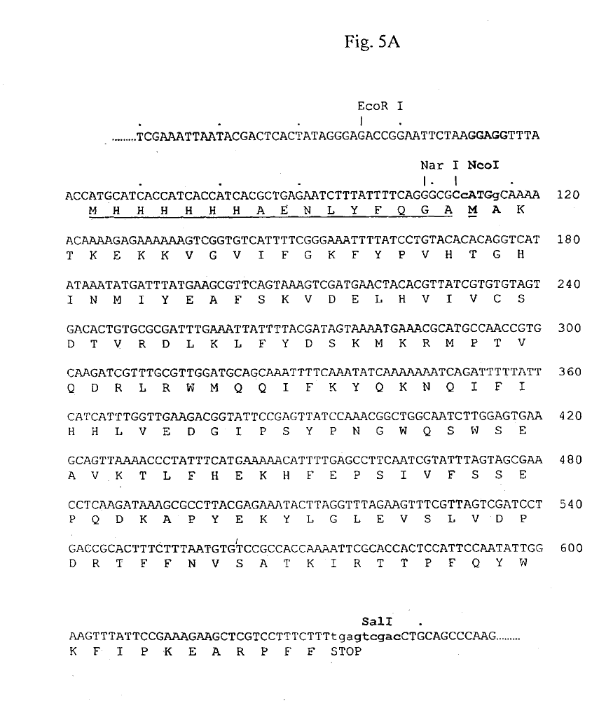

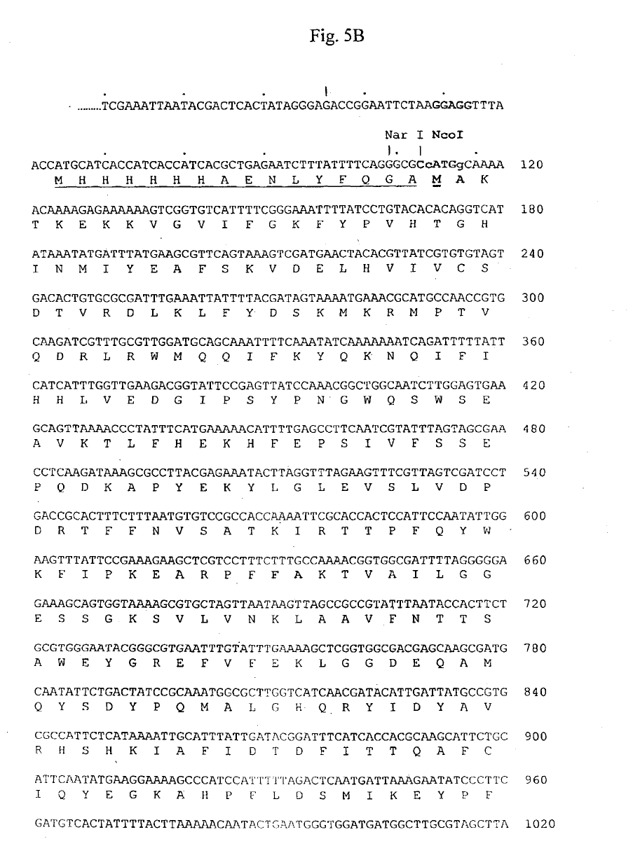

### DETAILED DESCRIPTION OF THE INVENTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. An isolated polynucleotide or polypeptide is one that is separated from the coding regions or encoded sequences with which it is contiguous in its original (e.g., natural) environment. An isolated polynucleotide could be part of a vector or a composition of matter, or could be contained within a cell, and still be “isolated” because that vector, composition of matter, or particular cell is not the original environment of the polynucleotide.

As used herein, a NRKse “polynucleotide” refers to a molecule having at least 95% sequence identity to a nucleic acid sequence encoding an NRKse domain contained in SEQ ID NOs: 1, 5, 7, 9, 11, 13, 15, 17, 19, 21, 23, 25, 27, 29, 31, 33, 35, 37, 39, or 41. For example, the NRKse polynucleotide can contain the nucleotide sequence of the full length sequence, as well as fragments, epitopes, domains. and variants of the nucleic acid sequence. Moreover, as used herein, a NRKse “polypeptide” refers to a molecule having the translated amino acid sequence generated from the polynucleotide as broadly defined.

A NRKse “polynucleotide” also includes those polynucleotides capable of hybridizing, under stringent hybridization conditions, to NRKse domain sequences contained in SEQ ID NOs: 1, 5, 7, 9, 11, 13, 15, 17, 19, 21, 23, 25, 27 29, 31, 33, 35, 37, 39, or 41, or the complement thereof. “Stringent hybridization conditions” refers to an overnight incubation at 42° C. in a solution comprising 50% formamide, 5× SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

Also contemplated are nucleic acid molecules that hybridize to the NRKse polynucleotides at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20× SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5× SSC).

Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The NRKse polynucleotide can be composed of any polyribonucleotide or polydeoxribonucleotide, which may be unmodified RNA or DNA or modified RNA or DNA. For example, NRKse polynucleotides can be composed of single-and double-stranded DNA, DNA that is a mixture of single- and double-stranded regions, single- and double-stranded RNA, and RNA that is mixture of single and double-stranded regions, hybrid molecules comprising DNA and RNA that may be single-stranded or, more typically, double-stranded or a mixture of single-and double-stranded regions. In addition, the NRKse polynucleotides can be composed of triple-stranded regions comprising RNA or DNA or both RNA and DNA. NRKse polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

NRKse polypeptides can be composed of amino acids joined to each other by peptide bonds or modified peptide bonds, i.e., peptide isosteres, and may contain amino acids other than the 20 gene-encoded amino acids. The NRKse polypeptides may be modified by either natural processes or by chemical modification techniques which are well known in the art. Such modifications are well described in basic texts and in more detailed monographs, as well as in a voluminous research literature. Modifications can occur anywhere in the NRKse polypeptide, including the peptide backbone, the amino acid side-chains and the amino or carboxyl termini. It will be appreciated that the same type of modification may be present in the same or varying degrees at several sites in a given NRKse polypeptide.

A NRKse polypeptide “having biological activity” refers to polypeptides exhibiting activity similar, but not necessarily identical to, an activity of a NRKse polypeptide as measured in a particular biological assay. The level of activity need not be identical to that of the wild-type NRKse polypeptide, but rather substantially similar to the wild-type NRKse polypeptide (i.e., the polypeptide will exhibit greater activity or not more than about 25-fold less and, preferably, not more than about ten-fold less activity, and most preferably, not more than about three-fold less activity relative to the wild-type NRKse polypeptide.)

Therefore, the NRKse domain sequences disclosed in SEQ ID NOs: 1-42 are sufficiently accurate and otherwise suitable for a variety of uses well known in the art and described further below. For instance the NRKse domain of SEQ ID NO: 1 is useful for designing nucleic acid hybridization probes that will detect NRKse domain nucleic acid sequence:, contained in SEQ ID NO: 1. These probes will also hybridize to nucleic acid molecules in biological samples, thereby enabling a variety of forensic and diagnostic methods of the invention. Similarly, polypeptides identified from the NRKse domain of SEQ ID NO: 2, for example, may be used to generate antibodies which bind specifically to NRKse.

The present invention also relates to the NRKse gene corresponding to SEQ ID NOs: 1-42. The NRKse gene can be isolated in accordance with known methods using the sequence information disclosed herein. Such methods include preparing probes or primers from the disclosed sequence and identifying or amplifying the NRKse gene from appropriate sources of genomic material.

Also provided in the present invention are orthologs of the NRKse polynucleotides and polypeptides set forth in SEQ ID NOs: 1-42. Orthologs can be identified and isolated by making probes or primers from the sequences provided herein and screening other species for the orthologous nucleic acid and/or polypeptide. For example, other bacterial species or eukaryotic species can be screened to identify and isolate an NRKse polynucleotide.

The NRKse polypeptides can he prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

NRKse polypeptides are provided in an isolated form, and preferably are substantially purified. A recombinantly produced version of a NRKse polypeptide can be substantially purified by using conventional methods. NRKse polypeptides also can be purified from natural or recombinant sources using antibodies of the invention raised against the NRKse protein in methods which are well known in the art.

Polynucleotide and Polypeptide Variants

The term “variant” refers to a polynucleotide or polypeptide differing from the NRKse polynucleotide or polypeptide, but retaining essential properties thereof. Generally, variants are overall closely similar, and, in many regions, identical to the exemplified NRKse polynucleotides or polypeptides.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the NRKse polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of an NRKse domain of SEQ ID NOs: 1, 5, 7, 9, 11, 13, 15, 17, 19, 21, 23, 25, 27, 29, 31, 33, 35, 37, 39, or 41, or any fragment specified as described herein.

As a practical matter, whether any particular nucleic acid molecule or polypeptide is at least 80%, 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al (Comp. App. Biosci. 6:227-245 (1990)). In a sequence alignment the query and subject sequences are both DNA sequences. An RNA sequence can be compared by converting U's to T's. The result of the global sequence alignment is in percent identity. Preferred parameters used in a FASTDB alignment of DNA sequences to calculate percent identify are: Matrix=Unitary. k-tuple=4, Mismatch Penalty=1, Joining Penalty=30, Randomization Group Length=0, Cutoff Score=1, Gap Penalty5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction should be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections need be made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 90%, 95%, 96%, 97%, 98% or 99% identical to, for instance, the amino acid sequences of the NRKse domain shown in SEQ ID NO: 2 can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence. whichever is shorter.

If the subject sequence is shorter than the query sequence due to N- or C-terminal deletions, not because of internal deletions, a manual correction should be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C- termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N and C-terminal ends of the subject sequence, as displayed in the FASTDB alignment, which are not matched/aligned with the query sequence are manually corrected for. No other manual corrections need be made for the purposes of the present invention.

The NRKse variants contain alterations in their sequences relative to the exemplified sequences. Especially preferred are polynucleotide variants containing alterations which produce silent substitutions, additions, or deletions, but which do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. NRKse polynucleotide variants can be produced for a variety of reasons, e.g. to optimize codon expression for a particular host.

Naturally occurring NRKse variants are included within the invention. Such variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the NRKse polypeptides. For instance, one or more amino acids can be deleted from the N-terminus or C-terminus of the protein without substantial loss of biological function.

Even if deleting one or more amino acids from the N-terminus or C-terminus of a polypeptide results in modification or loss of one or more biological functions, other biological activities may still be retained. For example, the ability of a deletion variant to induce and/or to bind to antibodies which recognize the protein will likely be retained when less than the majority of the residues of the protein are removed from the N-terminus or C-terminus. Whether a particular polypeptide lacking N or C-terminal residues of a protein retains such immunogenic activities can readily be determined by routine methods described herein and otherwise known in the art.

Thus, the invention further includes NRKse polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change. As the authors state, proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and lie; replacement of the hydroxyl residues Ser and Thr; replacement of the acidic residues Asp and Glu; replacement of the amide residues Asn and Gin, replacement of the basic residues Lys, AQr, and His; replacement of the aromatic residues Phe, Tyr, and Tip, and replacement of the small-sized amino acids Ala, Ser, Thr, Met, and Gly.

Besides conservative amino acid substitutions, variants of NRKse include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as in IgG Fc fusion region peptide, or leader or secretory sequence, or a sequence facilitating purification. Such variant polypeptides are deemed to be within the scope of those skilled in the art from the teachings herein.

For example, NRKse polypeptide variants containing amino acid substitutions of charged amino acids with other charged or neutral amino acids may produce proteins with improved characteristics, such as less aggregation.

Polynucleotide and Polypeptide Fragments

In the present invention, a “polynucleotide fragment” refers to a short polynucleotide having a nucleic acid sequence of an NRKse domain shown in SEQ ID NOs: 1, 5, 7, 9, 11, 13, 15, 17, 19, 21, 23, 25, 27, 29, 31, 33, 35, 37, 39, or 41. The short nucleotide fragments are at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from one of the nucleotide sequences shown herein. These nucleotide fragments are useful as diagnostic probes and primers as discussed herein. Of course, larger fragments (e.g., 50, 150, 500, 600, 1000 nucleotides) are preferred. Preferably, the fragments encode a polypeptide which has biological activity. More preferably, these polynucleotides can be used as probes or primers as discussed herein.

In the present invention, a “polypeptide fragment” refers to a short amino acid sequence of an NRKse domain contained in SEQ ID NOs: 2, 6, 8, 10, 12, 14, 16, 18, 20, 22, 24, 26, 28, 30, 32, 34, 36, 38, 40, or 42. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, and so forth to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the NRKse protein having a continuous series of deleted residues from the amino or the carboxy terminus, or both. For example, any number of amino acids, ranging from about 1-30, can be deleted from the amino terminus of the NRKse polypeptide. Similarly, any number of amino acids, ranging from about 1-30, can be deleted from the carboxy terminus of the NRKse protein. Furthermore, any combination of the above amino and carboxy terminus deletions are preferred. Similarly, polynucleotide fragments encoding these NRKse polypeptide fragments are also preferred.

Also preferred are NRKse polypeptide and polynucleotide fragments characterized by structural or functional domains. For example, such fragments may comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. Such preferred regions may include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Moreover, polynucleotide fragments encoding these domains are also included within the invention.

Other preferred fragments are biologically active NRKse fragments. Biologically active fragments are those exhibiting activity similar, but not necessarily identical, to an activity of the NRKse polypeptide. The biological activity of the fragments may include an improved desired activity, or a decreased undesirable activity. NRKse activity can be measured using conventional techniques.

Epitopes and Antibodies

In the present invention, “epitopes” refer to NRKse polypeptide fragments having antigenic or immunogenic activity in an animal, e.g., in a human. A preferred embodiment of the present invention relates to a NRKse polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (Sec, for instance, Geysen et al., Proc. Natl. Acad Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind to the epitope (See, for instance, Wilson et al., Cell 37:767-778 (1984); Sutcliffe, J. G. et al, Science 219:660-666 (1983).)

Similarly, immunogenic epitopes can be used to induce antibodies according to methods well known in the art. (See, for instance, Sutcliffe et al., supra; Wilson et al., supra; Chow, M. e t al., Proc. Natl. Acad. Sci. USA 82:910-914; and Bittle, F. J., et al., J. Gen. Virol. 66:2347-2354 (1985).) The immunogenic epitopes may be presented together with a carrier protein, such as an albumin, to an animal system (such as a rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (Mab) is meant to include intact molecules a, well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a Fab or other immunoglobulin expression library. Moreover, antibodies of the present invention include chimeric, single chain, and humanized antibodies.

Fusion Proteins

Any NRKse polypeptide can be used to generate fusion proteins. For example, the NRKse polypeptide, when fused to a second protein, can be used as an antigenic tag. Antibodies raised against the NRKse polypeptide can be used to indirectly detect the second protein by binding to the NRKse.

Examples of domains that can be fused to NRKse polypeptides include not only heterologous signal sequences, but also other heterologous functional regions. The fusion does not necessarily need to be direct, but may occur through linker sequences.

Moreover, fusion proteins may also be engineered to improve characteristics of the NRKse polypeptide. For instance, a region of additional amino acids, particularly charged amino acids, may be added to the N-terminus of the NRKse polypeptide to improve stability and persistence during purification from the host cell or subsequent handling and storage. Also, peptide moieties may be added to the NRKse polypeptide to facilitate purification. Such regions may be removed prior to final preparation of the NRKse polypeptide. The addition of peptide moieties to facilitate handling of polypeptides are familiar and routine techniques in the art.

Moreover, NRKse polypeptides, including fragments, and specifically epitopes, can be combined with parts of the constant domain of immunoglobulins (IgG), resulting in chimeric polypeptides. These fusion proteins facilitate purification and show an increased half-life in vivo.

Moreover, the NRKse polypeptides can be fused to marker sequences, such as a peptide which facilitates purification of NRKse. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al, Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al, Cell 37:767 (1984)).

Thus, any of these above fusions can be engineered using the NRKse polynucleotides or the polypeptides.

Vectors, Host Cells, and Protein Production

The present invention also relates to vectors containing the NRKse polynucleotide, host cells, and the production of polypeptides by recombinant techniques. The vector may be, for example, a phage, plasmid, viral, or retroviral vector. Retroviral vectors may be replication competent or replication defective.

In the latter case, viral propagation generally will occur only in complementing host cells.

NRKse polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The NRKse polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Exemplary vectors preferred for use in bacteria include pQE70, pQE60 and pQE-9. available from QIAGEN, Inc. pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene, and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation. transduction, infection, or other methods. Such methods are described in many standard laboratory manuals, such as Davis et al, Basic Methods In Molecular Biology (1986).

NRKse polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

NRKse polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the NRKse polypeptides may be glycosylated or may be non-glycosylated. In addition, NRKse polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

The NRKse polynucleotides identified herein can be used in numerous ways and with known techniques. For example, the polynucleotides can be used in combination with recombinant DNA technology to produce isolated NRKse polypeptides of the invention. Additionally, such polynucleotides can be used to overexpress NRKse in bacterial strains., and in other cells, to increase the pool of NAD. For example, overexpression of NRKse is desirable in cells (e.g., bacterial cells) used in the industrial production of bulk chemicals, e.g., amino acids (see, e.g., U.S. Pat. No. 5,830,716). Overexpression of NRKse in such cells increases the intracellular NAD/NADP pool, which may otherwise limit growth of such cells. Now that the polynucleotide sequence for NRKse has been identified, conventional cloning and expression methods can be used for overexpression of NRKse. Alternatively, the polynucleotides of the invention can be used as probes or primers in the identification of additional NRKse polynucleotides. Similarly, the polynucleotides can be used diagnostically to identify an organism containing the polynucleotide (e.g., to distinguish S. typhi from S. enteritidis).

NRKse polypeptides can be used in assays to test for one or more biological activities, e.g., the ability to phosphorylate NmR in the presence of ATP or a polyphosphate, to produce NMN. For example, NRKse polypeptides can be used in vitro in the production of NMN or nucleotide analogs, which can in turn be used in the production of NAD or analogs thereof. Such NAD analogs can be used, for example, as antimicrobial agents. Exemplary nucleoside analogs that can be produced using the NRKse polypeptides of the invention are disclosed in U.S. Pat. Nos. 5,700,786 and 5,569,650.

The NRKse polypeptides of the invention can be used in the production of phosphorylated forms of pyridine nucleotides. Exemplary analogs include 3′deazaguanosine and tiazofurin, which are anti-cancer prodrugs. Phosphorylated forms of these nucleoside analogs are needed for in vitro testing, since their mechanism of action in vivo is dependent upon phosphorylation inside the cell to produce the active drug (Se., e.g., Plunkett et al., Pharmacol Ther. 49:239-68 (1991)).

NRKse polypeptides may be used to screen for compounds that bind to NRKse. The binding of NRKse and the molecule may activate (agonist), increase, inhibit (antagonist), or decrease activity of the NRKse. Examples of such molecules include antibodies, oligonucleotides, proteins (e.g., receptors),or small molecules.

Preferably, the molecule is closely related to the natural ligand of NRKse, e.g., a fragment of the ligand, or a natural substrate, a ligand, a structural or functional mimetic. The molecule can be rationally designed using known techniques.

Preferably, the screening for these molecules involves producing appropriate cells which express NRKse, e.g., as a secreted protein or on the cell membrane. Preferred cells include cells from mammals, yeast, Drosophila, or bacteria such as E. coli. Cells expressing NRKse (or cell membrane containing the expressed polypeptide) are then preferably contacted with a test compound potentially containing the molecule to observe binding, stimulation, or inhibition of activity of either NRKse or the molecule.

The assay may simply test binding of a candidate compound to NRKse, wherein binding is detected by a label, or in an assay involving competition with a labeled competitor.

Alternatively, the assay can be carried out using cell-free preparations, polypeptide/molecule affixed to a solid support, chemical libraries, phage display libraries, or natural product mixtures. The assay may also simply comprise the steps of mixing a candidate compound with a solution containing NRKse, measuring NRKse/molecule activity or binding, and comparing the NRKse/molecule activity or binding to a standard.

The invention includes a method of identifying compounds which bind to NRKse comprising the steps of: (a) incubating a candidate binding compound with NRKse; and (b) determining if binding has occurred. Moreover the invention includes a method of identifying modulators, i.e., agonists/antagonists comprising the steps of: (a) incubating a candidate compound with NRKse, (b) assaying a biological activity, and (c) determining if a biological activity of NRKse has been altered.

Because NRKse is an essential enzyme in V-factor dependent Pasteurellaceae, e.g., V-factor dependent Haemophilus, compounds that bind to NRKse are candidate antibiotics, and can be used as lead compounds in the development of drugs for treating or inhibiting infections by pathogens of the Pasteurellaceae family. Compounds that inhibit NRKse activity are expected to be useful antibiotics. Compounds that inhibit both NRKse and NMNATse, which can be identified using methods described herein, are particularly preferred antibiotics. Compounds that inhibit NRKse and/or NMNATse activity can also be used to inhibit the growth or activity of other pathogens in which NRKse is not an essential enzyme, since most organisms have such NAD recycling machinery. When the compound is used to inhibit NRKse activity in an organism in which NRKse is not essential, such uses preferably are in combination with other antibiotics. Moreover, it is preferable that a compound used as an antibiotic preferentially inhibits the NRKse of the pathogen as compared to an NRKse of the host to be treated. Conventional techniques known to those skilled in the art, e.g., competition assays, can be used to identify compounds that preferentially inhibit the NRKse of the pathogen.

Thus, the polypeptides of the invention can be used to identify compounds that can be used to inhibit or treat infections by Gram-negative and Gram-positive bacterial families and fungi such as Mycobacterium tuberculosis, Pseudomonas aeruginosa, and any other pathogens which contain the NRKse of the present invention. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Emphysema, sepsis, Lyme Disease, Cat-Scratch Disease, Dysentary, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, and wound infections.

Having generally described the invention, the same will be more readily understood by reference to the following examples, which are provided by way of illustration and are not intended as limiting.

### EXAMPLES

### Example 1

### Cloning and Expression of NRKse

Materials and Experimental Procedures

E. coli strain DH5α (Gibco-BRL), Epicurian Coli BL21 and BL2 1 (DE3) (Stratagene) were used for cloning and expression. For expression of all genes in E. coli, a pET-derived vector containing a T7 promoter, a 6His-Tag and a TEV-protease cleavage site (such as described in Osterman, et al., Biochemistry, 33:13662-13667 (1994)), or a similar vector with a Trc promoter, e.g., pProEX HTa from Gibco BRL, was used.

For PCR amplification of the target genes, the total genomic DNA was obtained from ATCC (http://www.atcc.org/): Haemophilus influenzae Rd, KW20 (51907D at ATCC) Haemophilus influenzae, and Salmonella typhimurium were used. Human brain cDNA was purchased from Clontech. All NMN derivatives, ADH, other chemicals and biochemicals were purchased from Sigma-Aldrich-Fluka. Calf Intestinal Alkaline Phosphatase was obtained from Fermentas.

Enzymes for DNA manipulations were from NEB and Fermentas. For PCR, Pfu polymerase was purchased from Stratagene. Plasmid purification kits and Ni-NTA resin were purchased from Qiagen. Reagents for DNA sequencing were purchased from ABI. Oligonucleotides for PCR and sequencing were purchased from Sigma-Genosys.

An AKTA FPLC system and columns were obtained from Pharmacia. A Beckman spectrophotometer DU 640 with a thermostated 6-cuvette cell holder was used. A Gilson HPLC system consisting of a model 322 gradient pump, model 152 UV/V is detector and model 234 Autosampler HPLC system was used.

PCR Amplification and Cloning

Sequences encoding NRKse from H. influenzae (located in the C-terminal portion of NadR), NadR from S. typhimurium, and NMNATse from H. sapiens NadD2_HS) were amplified and cloned as follows. Primers used for PCR amplification of full-size coding regions and for the introduction of restriction sites are listed in Table 3. Reactions were performed according to conventional protocols. In each case, the production of a specific fragment was optimized by varying an annealing temperature. The following conditions were used:

| Fragment to be amplified: | Primer SEQ ID | Protocol |
|---|---|---|
| NadR_HI _N-domain | Nos. 43, 43 | 1 cycle: |
| | | 95° C.-3′ |
| | | 35 cycles: |
| | | 94° C.-45″ |
| | | 57° C.-45″ |
| | | 72° C.-90″Taq |
| NadR_HI_Truncated | Nos. 43, 45 | Same as above |
| NadR_SY | Nos. 46, 47 | 1 cycle: |
| | | 96° C.-3′ |
| | | 35 cycles: |
| | | 96° C.-60″ |
| | | 52° C.-60″ |
| | | 72° C.-90″Taq |
| NadD2_HS | Nos. 48, 49 | 1 cycle: |
| | | 94° C-1′ |
| | | 35 cycles: |
| | | 94° C.-30″ |
| | | 68° C-3′ |
| | | “Advantage” Kit |
| | | Clontech |

PCR fragments were purified, digested with enzymes, and cloned (using standard protocols) into an expression vector, which was cleaved by NcoI and SalI. Selected clones were verified by DNA sequence analysis.

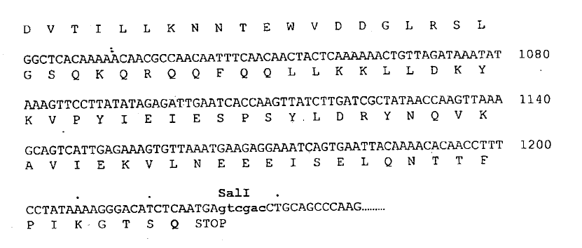

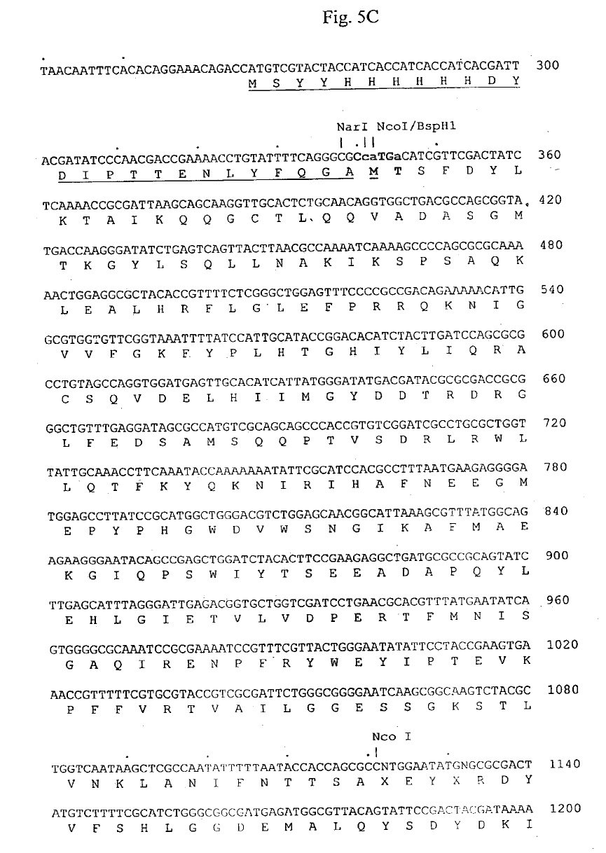

The expression constructs were designed as translational fusions of a target gene with a 6×His-tag and a cleavage site for TEV-protease. All clones were verified by DNA sequencing of both strands. No mutations compared to the original sequence were found. The sequence of a portion of each constrict containing the coding region is shown in FIGS. 5A-5D.

Expression and Purification

All proteins were expressed as N-terminal fusions with a 6×His tag and a TEV-protease cleavage site. Transcription was initiated by T7 polymerase, which inmost cases was achieved by IPTG induction in E. coli strain BL21/DE3. Cells were grown to OD₆₀₀=0.8-1.0 at 37° C. in LB medium (in 50 mL for analytical purposes or in 6L for preparative purification). IPTG was added to provide a final concentration of 0.2 mM, and cells were harvested after 6-12 hours of incubation while shaking at 20° C.

Expression analysis and protein purification were performed using standard techniques, as described by Osterman et al., in J Biol. Chem. 270, 11797-802 (1995) for ornithine decarboxylase expressed in the same vector. Briefly, harvested cells were resuspended in A-buffer (20 mM HEPES, pH 7, containing 100 mM NaCl, 0.03% Brij-35, 2 mM β-mercaptoethanol) with 2 mM PMSF and a protease inhibitors cocktail (Sigma). Lysozyme was added to a concentration of 1 mg/mL. After 20 minutes of incubation on ice, the cell suspension was frozen in liquid nitrogen. After thawing and sonication, cell debris was removed by centrifugation at 20,000 rpm for 2 hours. Tris-HCl buffer, pH 8, was added to the supernatant up to a final concentration of 50 mM, and the supernatant was loaded onto a Ni-NTL agarose column.

For analysis of gene expression, micro bio-spin columns (Bio-Rad) with 50 μL of Ni-NTA agarose were used. All loading and washing steps were performed using gravity flow. The microcolumns were washed with 5 column volumes of A-buffer, and 10 volumes of A-buffer containing 1M NaCl and 0.3% Brij-35. Elution was performed with 300 μL of A-buffer containing 200 mM imidazole. To estimate the distribution of expressed proteins between the soluble fraction and inclusion bodies, insoluble material was extracted from cell debris using 2 mL of A-buffer containing 8M urea. Partial protein purification was performed using the same microcolumns and the same procedure, except that all of the solutions were prepared in 8M urea. Both samples (i.e., protein purified under native and denaturing conditions) were analyzed by SDS-PAGE in 12% minigels (using MiniProtean 3, Bio-Rad), and compared to negative controls (empty vector or/and non-induced culture).

For preparative purification of the proteins, a 5-15 mL Ni-NTA agarose column was used. The volume was chosen depending on the expression level estimated in a small aliquot as described above. After loading and washing using a peristaltic pump and buffers as above. a gradient elution with imidazole (0-200 mM in buffer A) was performed using the FPLC system. Column fractions were analyzed by SDS-PAGE, pooled, and concentrated to a final volume of 2-20 mL, to provide a protein concentration 5-50 mg/mL depending on the protein and the expression level. Aliquots of 1 or 2 mL of the concentrated samples (containing additionally 5 mM DTT and 2 mM EDTA) were loaded onto a HiLoad Superdex 200 16/60 column (Pharmacia). Gel-filtration was performed in 50 mM Tris-HCl or HEPES buffer pH 7.5, containing 100 mM NaCl, 0.5 mM EDTA and 1 mM DTT, using FPLC. Fractions containing the biologically active protein were pooled together, concentrated to 1-50 mg/mL, and aliquots were frozen in liquid nitrogen and stored at −80° C.

Expression and Purification of Human NMNATse (NadD2_HS) for use in a Coupled Assay

As described herein, NMNATse was coupled with NRKse in an assay to measure NAD production and conversion to NADH. The DNA and protein sequences of NMNATse (NadD2) from Homo sapiens are set forth in FIG. 5D The PCR primers listed in Table 3 were designed based on the human cDNA sequence. Cloning, expression analysis, and large scale expression and purification were performed as described above for NadR proteins. After gel-filtration, NadD2_HS was concentrated to ˜0.58 mg/mL, and tested in a NMNATse assay described above. Specific activity on NMN was ˜50 un/mg.

Synthesis of NRKse Substrate(s)

Nicotinamide Ribonucleoside (NmR) and Nicotinate Ribonucleoside (NaR) were produced by Alkaline Phosphatase (EC 3.1.3.1; Fermentas) treatment of 1 mM solutions of NMN and NaMN, respectively at 37° C. for about 12 hrs in 200 mM Tris/HCl, pH 8.0 and 10 mM MgCl₂ (as described in Godek et al., Antimicrob. Agents Chemother. 34, 1473-9 (1990)). The completion of the reaction was monitored by HPLC as described below.

HPLC Assay

An HPLC assay was carried out using Gilson HPLC system consisting of model 322 gradient pump, model 152 UV/V is detector and model 234 Autosampler. IBM-PC compatible computer, equipped with model 506 interface and UniPort software was used for system control and data collection. The system was equipped with a 50+4.6(ID) mm C18 column (Supelco).

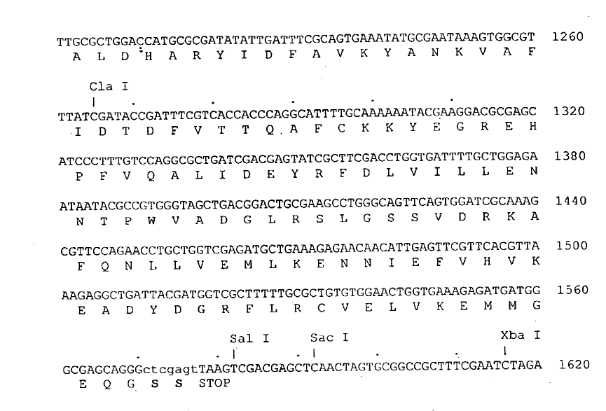

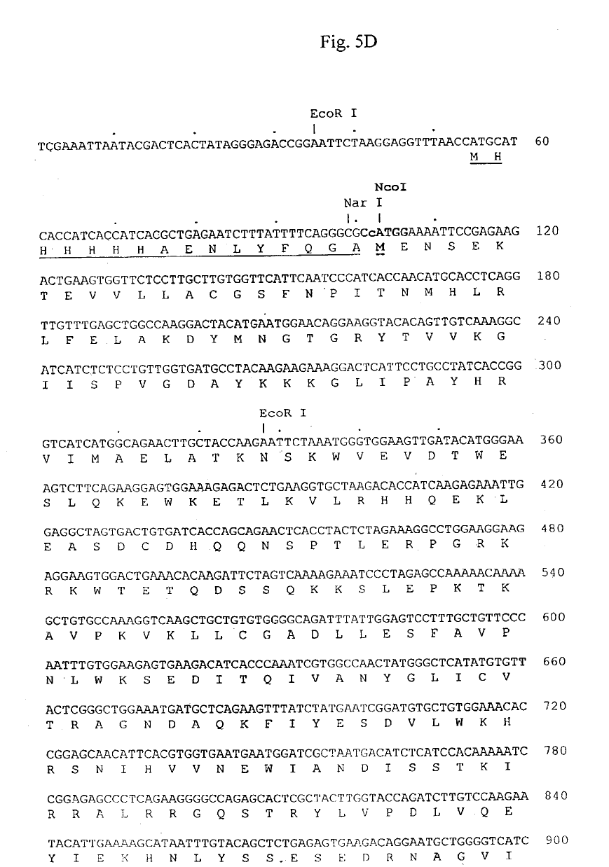

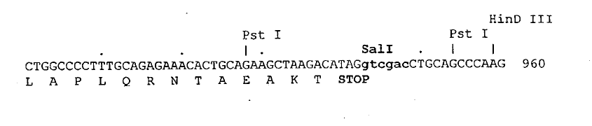

HPLC separation was carried out in isocratic mode with eluent containing 50 mM sodium phosphate (pH 5.5), 8 mM tetrabutylammonium bromide and 8% of methanol. The eluent flow rate was 1 ml/min, and detection was carried out at 254 nm. As shown in FIG. 6, treatment of NmR with NadR in the presence of ATP leads to the production of NMN and ADP, confirming that the sequence encoding NRKse is contained within NadR.

NMNATse and NRKse Assays

The discontinuous spectral NMNATse assay procedure (used for both human NadD2_HS and NadR) coupled to alcohol dehydrogenase (ADH) catalyzed conversion of NAD to NADH was adapted from Balducci, E., et al., Biochem. J., 310:395-400 (1995), as described in U.S. Ser. No. 09/550,398, filed Apr. 14, 2000. The assay was performed in disposable UV-transparent plastic cuvettes in a 6-cuvette autosampler of Beckman DU-640 set at 37° C. The 500 μL mixture contained 100 mM HEPES, pH 7.5, 115 mM ethanol, 40 mM semicarbazide, 2 mM ATP, 3 units of ADH (Sigma), and 0.2-2 milliunits of NMNATse (0.1-10 μg depending on specific activity). A reaction was initiated by adding 10 μL of 50 mM NMN, and monitored at 340 nm over 20 minutes. An extinction coefficient of NADH equal to 6.22 mM⁻¹ cm⁻¹ was used for rate calculations. One unit of enzyme was defined as a quantity capable of producing 1 micromole of NADH per minute. The results of this assay are set forth in Table 2.

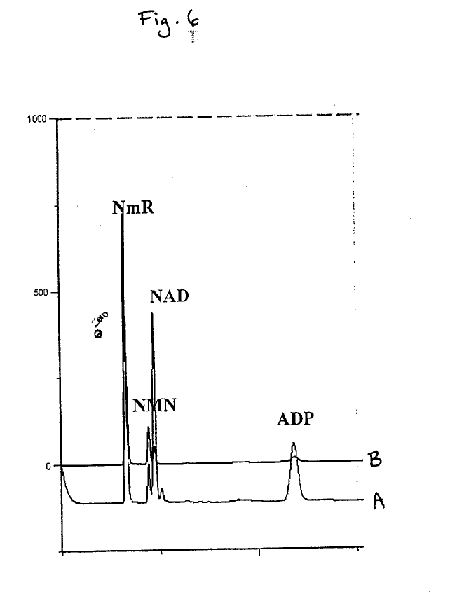

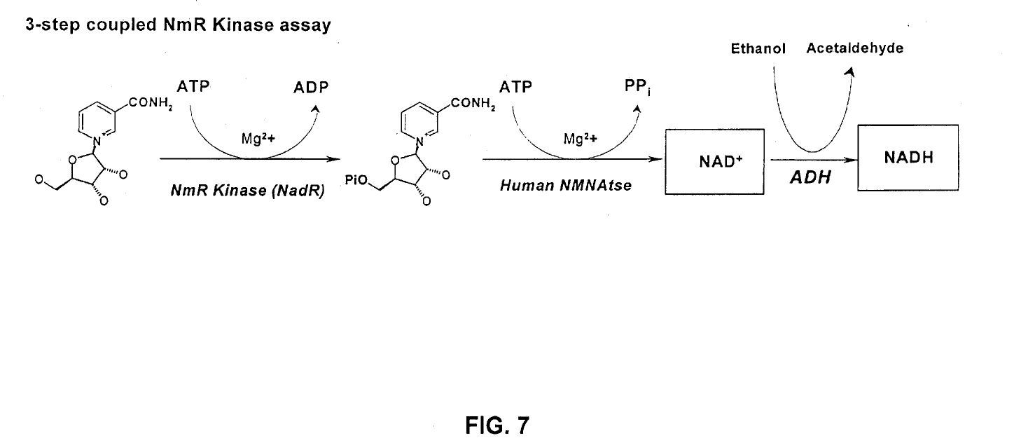

The discontinuous spectral NRKse assay was based on enzymatic coupling of NmR→NMN conversion to production of NAD, catalyzed by excess amounts of recombinant purified NadD2_HS enzyme (NMNATse). NAD production was further coupled to NADH conversion by ADH, as in the NMNATse assay described above. A schematic representation of this assay is set forth in FIG. 7.

The assay was performed in disposable UV-transparent plastic cuvettes in a 6-cuvette autosampler of Beckman DU-640 set at 37° C. The 500 μL mixture contained 100 mM Tris/HCl pH 8.0, 115 mM ethanol, 40 mM semicarbazide, 0.8 mM NmR, 0.15 units of NadD2_HS, 3 units of ADH (Sigma), and 0.2-2 milliunits of NRKse (0.2-2 μg, depending on the specific activity). A reaction was initiated by adding 50 μL of 50 mM ATP, and monitored at 340 nm over 20 minutes. An extinction coefficient of NADH equal to 6.22 mM⁻¹ cm⁻¹ was used for rate calculations. One unit of enzyme was defined as a quantity capable of producing 1 micromole of NADH per minute. The results of this assay are set forth in Table 2.

Together, the NMNATse and NRKse assays show that both NadR from H. influenzae and S. typhi possess NRKse activity in addition to NMNATse activity. NMNATse activity is located in the N-terminal domain of NadR. NRKse activity is located in the C-terminal domain of NadR. The first 38 amino acids of NadR of H. influenzae is not relevant for either type of activity, and Met-38 is a possible translation start site.

These assays can readily be adapted for high throughput screening of compounds that modulate NMNATse and/or NRKse activity, as further described herein. For example, chemical compound libraries, phage display libraries, and the like, can readily be screened using 96- or 384-well plates and a plate reader in lieu of a cuvette.

Additional, prophetic, examples are provided below.

### Example 2

### Bacterial Expression of NRKse in Recombined Bacterial Cells

To synthesize insertion fragments, a NRKse polynucleotide encoding a NRKse polypeptide of the invention is amplified using PCR oligonucleotide primers comprising the 5′ and 3′ ends of the NRKse domain of the DNA sequence shown as SEQ ID NO: 2, 4, 6, 8, 10, 12, 14, 16, 18, 20, 22, 24, 26, 28, 30, 32, 34, 36, 38, 40, or 42. The primers used to amplify the DNA insert preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

Specifically, to clone the NRKse protein in a bacterial vector, both a 5′ and 3′ primer of a sequence specific to the terminal regions of an NRKse sequence disclosed herein is designed. The point in the protein coding sequence where the 5′ primer begins may be varied to amplify a DNA segment encoding any desired portion of the complete NRKse protein shorter or longer than the full-length protein. Moreover, described primers could then be used to amplify the corresponding NRKse nucleotide sequence by PCR methodology—taking advantage of restriction sites present within the bacterial vector and added to the terminal ends of the corresponding NRKse-specific primers.

For example, the pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen. Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells arc grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 minutes at 600×g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified NRKse protein is then renatured by dialyzing it against phosphate-buffered saline (PBS) or 50 mM Na-acetate, pH 6 buffer plus 200 mM NaCl. Alternatively, the NRKse protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified NRKse protein is stored at 4° C. or frozen at −80° C.

### Example 3

### Purification of NRKse Polypeptide from an Inclusion Body

The following alternative method can be used to purify NRKse polypeptide expressed in bacteria when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm. On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the NRKse polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant NRKse polypeptide should exhibit greater than 95% purity after the above refolding and purification steps. No major contaminant bands should be observed from Coomassie blue stained 16% SDS-PAGE gel when 5 μg of purified protein is loaded.

### Example 4

### Cloning and Expression of NRKse in a Baculovirus Expression System

Orthologs of the NRKse sequences set forth herein are included within the invention. For example, eukaryotic, e.g., mammalian, NRKse polynucleotides and polypeptides are included. Such orthologs can be expressed in a baculovirus expression system, if desired.

In this example, the plasmid shuttle vector pA2 is used to insert NRKse polynucleotide into a baculovirus to express NRKse. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned NRKse polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, an NRKse nucleotide sequence including an AUG initiation codon is amplified using PCR. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment hen is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five fig of a plasmid containing, the polynucleotide is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One μg of BaculoGold™ virus DNA and 5 μg of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced NRKse protein.

### Example 5

### Expression of NRKse in Mammalian Cells

NRKse polypeptides, particularly a mammalian orthologs, can be expressed in a mammalian cell, if desired. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C 127 cells, Cos 1, Cos 7 and CV1, quail QC1-3 cells, mouse L, cells and Chinese hamster ovary (CHO) cells.

Alternatively, NRKse polypeptide can be expressed in stable cell lines containing the NRKse polynucleotide integrated into a chromosome. The co-transfection with a selectable marker such as DHFR, GPT, neomycin, hygromycin allows the identification and isolation of the transfected cells.

The transfected NRKse gene can also be amplified to express large amounts of the encoded protein. The DHFR (dihydrofolate reductase) marker is useful in developing cell lines that can y several hundred or even several thousand copies of the gene of interest. (See, e.g., Alt, F. W., et al. J Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer. (Boshart et al., Cell 41:521-530 (1985).) Multiple cloning sites, e.g., with the restriction enzyme cleavage sites BamHI, XbaI and Asp718, facilitate the cloning of NRKse. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

The NRKse polynucleotide cam be amplified according to conventional protocols, and as described herein. The amplified fragment is isolated from a 1% agarose gel using a commercially available kit (“Geneclean,” BIO 101 Inc., La Jolla, Cailf.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligaited with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of NRKse is analyzed, for instance, by SDS-PAGE and Western blot or by HPLC analysis.

### Example 6

### Construction of N-Terminal and/or C-Terminal Deletion Mutants

The following general approach may be used to clone a N-terminal or C-terminal deletion mutant. Generally, two oligonucleotide primers of about 15-25 nucleotides are derived from the desired 5′ and 3′ positions of a polynucleotide disclosed herein. The 5′ and 3′ positions of the primers are determined based on the desired NRKse polynucleotide fragment. An initiation and stop codon are added to the 5′ and 3′ primers respectively, if necessary, to express the NRKse polypeptide fragment encoded by the polynucleotide fragment.

Additional nucleotides containing restriction sites to facilitate cloning of the NRKse polynucleotide fragment in a desired vector may also be added to the 5′ and 3′ primer sequences. The NRKse polynucleotide fragment is amplified from genomic DNA using the appropriate PCR oligonucleotide primers and conditions discussed herein or known in the art. The NRKse polypeptide fragments encoded by the NRKse polynucleotide fragments of the present invention may be expressed and purified in the same general manner as the full length polypeptides, although routine modifications may be necessary due to the differences in chemical and physical properties between a particular fragment and full length polypeptide.

### Example 7

### Protein Fusions of NRKse

NRKse polypeptides can be preferably fused to other proteins. These fusion proteins can be used for a variety of applications. For example, fusion of NRKse polypeptides to His-tag, HA-tag, protein A, IgG domains, and maltose binding protein facilitates purification. (See EP A 394,827; Traunecker, et al., Nature 331:84-86 (1988).) Similarly, fusion to IgG-1, IgG-3, and albumin increases the half-life time in vivo. Nuclear localization signals fused to NRKse polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. Methods for producing such fusion proteins are well known in the art.

### Example 8

### Production of an Antibody

The antibodies of the present invention can be prepared by a variety of methods. (See, Current Protocols, Chapter 2.) For example, cells expressing NRKse are administered to an animal to induce the production of sera containing polyclonal antibodies. In a preferred method, a preparation of NRKse protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with NRKse polypeptide or with a secreted NRKse polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the NRKse polypeptide.

Alternatively, additional antibodies capable of binding to NRKse polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the NRKse protein-specific antibody can be blocked by NRKse. Such antibodies comprise anti-idiotypic antibodies to the NRKse protein-specific antibody and can be used to immunize an animal to induce formation of further NRKse protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted NRKse protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi el al., BioTechniques 4:214 (1986); Cabilly et al, U.S. Pat. No. 4,816,567; Taniguchi et al. EP 171496; Morrison et al., EP 173494; Neuberger et al., WO 8601533; Robinson et al., WO 8702671; Boulianne et al., Nature 312:643 (1984); Neuberger et al., Nature 314:268 (1985)).

### REFERENCES

1. Amici, A. et al. (1997)FEBS Lett419, 263-7

2. Atkinson, W. H. and Winkler. H. H. (1989) J Bacteriol 171, 761-6

3. Balducci, E., Emanuelli, M., Raffaelli, N. and Magni, G. (1989) Boll Soc Ital Biol Sper 65, 1059-66.

4. Balducci, E. et al. (1992) Biochem Biophys Res Commun 189, 1275-9

5. Balducci, E. et al. (1995) Biochem J 310, 395-400

6. Balducci, E. et al. (1995) Anal Biochem 228, 64-8

7. Begley, T. P. et al. (1999) Curr Opin Chem Biol 3, 623-9

8. Begley, T. P. et al. (1999) Arch Microbiol 171, 293-300

9. Begley, T. P. et al. (2001) Vitan Horm 61, 103 -19

10. Bork, P., Holm, L., Koonin, E. V. and Sander, C. (1995) Proteins 22, 259-66

11. Boshoff, H. I. and Mizrahi, V. (1998) J Bacteriol 180, 5809-14

12. Boulton, S., Kyle, S. and Durkacz, B. W. (1997) Br J Cancer 76, 845-51

13. Brunngraber, E. F. and Chargaff, E. (1977) Proc Natl Acad Sci USA 74, 4160-2.

14. Chestukhina, G. G. et al. (19910) J Protein Chem 9, 501-7

15. Cole, S. T. et al. (1998) Nature 393, 537-44

16. Colovos, C., Cascio, D. and Yeates, T. O. (1998) Structure 6, 1329-37

17. Cordwell, S. J. (1999) Arch Microbiol 172, 269-79

18. Curnow, A. W. et al. (1997) Proc Natl Acad Sci USA 94, 11819-26

19. Cynamon, M. H., Sorg, T. B. and Patapow, A. (1988) J Gen Microbiol 134, 2789-99.

20. D'Angelo, I. et al. (2000) Structure Fold Des 8, 993-1004

21. Dahmen, W., Webb, B. and Preiss, J. (1967) Arch Biochem Biophys 120, 440-50

22. Das, S. et al. (2000) FEMS Microbiol Lett 190, 87-91.

23. Daugherty, M., Vonstein, V., Overbeek, R. and Osterman, A. (2001) J Bacteriol 183, 292-300.

24. Denicola-Seoane, A. and Anderson, B. M. (1990) J Gen Microbiol 136, 425-30

25. Efimov, I., Kuusk, V., Zhang, X. and McIntire, W. S. (1998) Biochemistry 37, 9716-23

26. Eisenstein, E. and Beckett, D. (1999) Biochemistry 38, 13077-84

27. Emanuelli, M. et al. (1992) Arch Biochem Biophys 298, 29-34

28. Emanuelli, M. et al. (1995) Biochem Pharmacol 49, 575-9

29. Emanuelli, M. et al. (1999) FEBS Lett 455, 13-7

30. Emanuelli, M. et al. (2000) J Biol Chem

31. Foster, J. W. and Moat, A. G. (1978) J Bacteriol 133, 775-9

32. Foster, J. W., Kinney, D. M. and Moat, A. G. (1979) J Bacteriol 138, 957-61

33. Foster, J. W., Kinney, D. M. and Moat, A. G. (1979) J Bacteriol 137, 1165-75

34. Foster, J. W. and Moat, A. G. (1980) Microbiol Rev 44, 83-105.

35. Foster, J. W. et al. (1990) J Bacteriol 172, 4187-96

36. Foster, J. W. and Penfound, T. (1993) FEMS Microbiol Lett 112,179-83

37. Frothingham, R. et al. (1996) Antimicrob Agents Chemother 40, 1426-31

38. Gaida, A. V., Osterman, A. L., Rudenskaia, G. N. and Stepanov, V. M. (1981) Biokhimiia 46, 181-9

39. Geerlof, A., Lewendon, A. and Shaw, W. V. (1999) J Biol Chem 274, 27105-11

40. Gehring, A. M., Mori, I. and Walsh, C. T. (1998) Biochemistry 37, 2648-59

41. Godek, C. P. and Cynamon, M. H. (1990) Antimicrob Agents Chemother 34, 1473-9.

42. Goupil, N., Corthier, G., Ehrlich, S. D. and Renault, P. (1996) Appl Environ Microbiol 62, 2636-4.0

43. Grishin, N. V., Osterman, A. L., Goldsmith, E. J. and Phillips, M. A. (1996) Proteins 24, 272-3

44. Grishin, N. V. et al. (1999) Biochemistry 38, 15174-15184

45. Gross, J. W., Rajavel, M. and Grubmeyer, C. (1998) Biochemistry 37, 4189-99

46. Guo, M., Sun, Z. and Zhang, Y. (2000) J Bacteriol 182, 3881-3884

47. Hughes, K. T., Ladika, D., Roth, J. R. and Olivera, B. M. (1983) J Bacteriol 155, 213-21

48. Hughes, K. T. et al (1983) J Bacteriol 154, 1126-36

49. Hughes, K. T., Olivera, B. M. and Roth, J. R. (1988) J Bacteriol 170, 2113-20

50. Izard, T. and Geerlof, A. (1999) Embo J 18, 2021-30

51. Izard, T., Geerlof, A., Lewendon, A. and Barker, J. J. (1999) Acta Crystallogr D Biol Crystallogr 55, 1226-8

52. Jackson, L. K. et al. (2000) Biochemistry 39, 11247-57.

53. Kahn, D. W. and Anderson, B. M. (1986) J Biol Chem 261, 6016-25.

54. Kasarov, L. B. and Moat, A. G. (1973) J Bacteriol 115,35-42

55. Kasarov, L. B. and Moat, A. G. (1973) Biochim Biophys Acta 320, 372 -8.

56. Kinney, D. M., Foster, J. W. and Moat, A. G. (1979) J Bacteriol 140, 607-11

57. Kobayashi, M. et al. (1997) Proc Natl Acad Sci USA 94, 11986-91

58. Koonin, E. (1999) Trends Genet 15, 131

59. Liu, G., Foster, J., Manlapaz-Ramos, P. and Olivera, B. M. (1982) J Bacteriol 152, 1111-6

60. Ludwig, A. et al. (1990) Cancer Res 50, 2470-5.

61. Luque, I., Flores, E. and Herrero, A. (1993) Plant Mol Biol 21, 1201-5

62. Magni, G. et al. (1997) Methods Enzymol 280, 248-55

63. Magni, G. et al. (1997) Methods Enzymol 280, 241-7

64. Magni, G. et al. (1999) Adv Enzymol Relat Areas Mol Biol 73, 135-82

65. Martin, P. R., Shea, R. J. and Mulks, M. H. (2001) J Bacteriol 183, 1168 -1174.

66. Meyer, C. R. et al. (1998) Arch Biochem Biophys 353, 152-9

67. Meyer, C. R., Yirsa, J., Gott, B. and Preiss, J. (1998) Arch Biochem Biophys 352, 247-54

68. Miflin, J. K. et al. (1995) Avian Dis 39, 304-8.

69. Moat, A. G. and Foster, J. W. (1987) in Pyridine Nucleotide Coenzymes Part B (Dolphin D, P. R., Avramovich O, ed.), pp. 1-20, John Wiley & Sons

70. Mushegian, A. (1999) J Molec. Microbiol. Biotechnol. 1, 127-128

71. Natalini, P., Ruggieri, S., Raffaelli, N. and Magni, G. (1986) Biochemistry 25, 3725-9

72. Niven, D. F. and O'Reilly, T. (1990) Int J Syst Bacteriol 40, 1-4.

73. Nord, L. D., Stolfi, R. L., Colofiore, J. R. and Martin, D. S. (1996) Biochem Pharmacol 51, 621-7.

74. Novo, C., Tata, R., Clemente, A. and Brown, P. R. (1995) FEBS Lett 367, 275-9

75. O'Handley, S. F., Frick, D. N., Dunn, C. A. and Bessman, M. J. (1998) J Biol Chem 273, 3192-7

76. O'Reilly, T. and Niven, D. F. (1986) Can J Microbiol 32, 733-7.

77. O'Reilly, T. and Niven, D. F. (1986) J Gen Microbiol 132, 807-18.

78. Osterman, A. L. et al. (1984) Biokhimiia 49, 292-301

79. Osterman, A. L. et al. (1992) J Protein Chem 11, 561-70

80. Osterman, A., Grishin, N. V., Kinch, L. N. and Phillips, M. A. (1994) Biochemistry 33, 13662-7

81. Osterman, A. L. et al. (1995) Biochemistry 34, 13431-6

82. Osterman, A. L., Kinch, L,. N.. Grishin, N. V. and Phillips, M. A. (1995) J Biol Chem 270, 11797-802

83. Osterman, A. L., Brooks, H. B., Rizo, J. and Phillips, M. A. (1997) Biochemistry 36, 4558-67

84. Osterman, A. L. et al. (1999) Biochemistry 38, 11814-26

85. Ostrowski, J. et al. (1989) J Biol Chem 264, 15796-808

86. Overbeek, R. et al. (1999) Proc Natl Acad Sci U S A 96, 2896-901

87. Park, U. E., Roth, J. R. and Olivera, B. M. (1988) J Bacteriol 170, 3725

88. Park, Y. S. et al. (1997) J Biol Chem 272, 15161-6

89. Penfound, T. and Foster, J. W. (1996) in Escherichia Coli and Salmonella pp. 721-730, ASM

90. Penfound, T. and Foster, J. W. (1999) J Bacteriol 181, 648-55

91. Phalip, V., Lemoine, Y. and Jeltsch, J. M. (1999) Curr Microbiol 39, 48-0350

92. Phalip, V., Kuhn, I., Lemoine, Y. and Jeltsch, J. M. (1999) Gene 232, 43-51

93. Raffaelli, N. et al. (1994) FEBS Lett 355, 233-6

94. Raffaelli, N. et al. (1997) J Bacteriol 179, 7718-23

95. Raffaelli, N. et al. (1999) FEBS Lett 444, 222-6

96. Raffaelli, N. et al. (1999) J Bacteriol 181, 5509-11

97. Rajavel, M., Lalo, D., Gross, J. W. and Grubmeyer, C. (1998) Biochemistry 37, 4181-8

98. Raynaud, C. et al. (1999) Microbiology 145, 1359-67

99. Reidl, J. et al. (2000) Mol Microbiol 35, 1573-81.

100. Romao, M. J. et al. (1992) J Mol Biol 226, 1111-30

101. Rudenskaia, G. N., Osterman, A. L. and Stepanov, V. M. (1980) Biokhimiia 45, 710-7

102. Ruggieri, S. et al. (1988) Experientia 44, 27-9.

103. Rusnak, F. et al. (1990) Biochemistry 29, 1425-35

104. Sasiak, K. and Saunders, P. P. (1996) Arch Biochem Biophys 333, 414-8.

105. Saunders, P. P., Tan, M. T., Spindler, C. D. and Robins, R. K. (1989) Cancer Res 49, 6593-9.

106. Scorpio, A. and Zhang, Y. (1996) Nat Med 2, 662-7

107. Shadenkov, A. A. et al. (1993) Mol Biol (Mosk) 27, 952-9

108. Shram, S. I. et al (1999) Prikl Biokhim Mikrobiol 35, 638-46.

109. Smulevitch, S. V. et al. (1991) FEBS Lett 293, 25-8

110. Smulevitch, S. V. et al. (1991) FEBS Lett 291, 75-8

111. Spector, M. P., Hill, J. M., Holley, E. A. and Foster, J. W. (1985) J Gen Microbiol 131, 1313-22.

112. Stepanov, V. M. et al. (1981) Biochem Biophys Res Commun 100, 1680-7

113. Sun, Z. and Zhang, Y. (1999) Antimicrob Agents Chemother 43, 537-42

114. Sunagawa, M., Okada, M., Gito, S. Y. and Hisamitsu, T. (2000) Masui 49, 250-4

115. Swanson, T. et al. (1998) Biochemistry 37, 14943-7

116. Swindell, S. R. et al. (1996) Appl Environ Microbiol 62, 2641-3

117. Teplyakov, A. et al. (1992) Eur J Biochem 208, 281-8

118. Tirgari, S., Spector, M. P. and Foster, J. W. (1986) J Bacteriol 167, 1086-1088.

119. Tiurin, S. A. et al. (1987) Biokhimiia 52, 918-26

120. van Heeswijk, W. C., Rabenberg, M., Westerhoff, H. V. and Kahn, D. (1993) Mol Microbiol 9, 443-57

121. Venkatachalam, K. V., Akita, H. and Strott, C. A. (1998) J Biol Chem 273, 19311-20

122. Venkatachalam, K. V., Fuda, H., Koonin, E. V. and Strott, C. A. (1999) J Biol Chem 274, 2601-4

123. Vinitsky, A. and Grubmeyer, C. (1993) J Biol Chem 268, 26004-10

124. Windsor, H. M., Gromkova, R. C. and Koornhof, H. J. (1991) J Gen Microbiol 137, 2415-21.

125. Windsor, H. M., Gromkova, R. C. and Koornhof, H. J. (1993) Int J Syst Bacteriol 43, 799-804.

126. Wise, D. J., Anderson, C. D. and Anderson, B. M. (1997) Vet Microbiol 58, 261-76.

127. Wolf, Y. I., Aravind, L. and Koonin, E. V. (1999) Trends Genet 15, 173-5

128. Worrall, D. M. and Tubbs, P. K. (1983) Biochem J 215, 153-7

129. Wu, M. X. and Preiss, J. (1998) Arch Biochem Biophys 358, 182-8

130. Zajc, A., Romao, M. J., Turk, B. and Huber, R. (1996) J Mol Biol 263, 269-83

131. Zalkin, H. (1985) Methods Enzymol 113, 263-4

132. Zalkin, H. (1985) Methods Enzymol 113, 303-5

133. Zalkin, H. (1985) Methods Enzymol 113, 293-7

134. Zalkin, H. (1985) Methods Enzymol 113, 282-7

135. Zalkin, H. (1985) Methods Enzymol 113, 273-8

136. Zalkin, H. (1985) Methods Enzymol 113, 297-302

137. Zalkin, H. (1985) Methods Enzymol 113, 278-81

138. Zalkin, H. (1985) Methods Enzymol 113, 287-92

139. Zalkin, H. (1985) Methods Enzymol 113, 264-73

140. Zalkin, H. (1997) Methods Enzymol 281, 214-8

141. Zhang, Y., Scorpio, A., Nikaido, H. and Sun, Z. (1999) J Bacteriol 181, 2044-9

142. Zhu, N., Olivera, B. M. and Roth, J. R. (1988) J Bacteriol 170, 117-25.

143. Zhu, N., Olivera, B. M. and Roth, J. R. (1989) J Bacteriol 171, 4402-9.

144. Zhu, N., Olivera, B. M. and Roth, J. R. (1991) J Bacteriol 173, 1311-20.

145. Zhu, N. and Roth, J. R. (1991) J Bacteriol 173, 1302-10.

All patents, patent applications, and references cited herein are incorporated herein by reference.

TABLE 1A
NAD/NADP biosynthetic genes in genomes containing NadR homologs

| | C | | | | | | | | | | | | |
|---|---|---|---|---|---|---|---|---|---|---|---|---|---|
| | | | | | Transport, Regulation, NMN/NAD/NADP | | | | | | | | |
| | | | | | Salvage | | | | | | | | |
| | | | | | Nad R, multifunctional | | | Niacin utilization | | | | | |
| | De novo biosynthesis | | | | protein: transcription | | NADP | | | Nicotinamide | | | |
| | | | Quinolinate | | regulator, nmn | | phosphatase | | Nicotinate | Nicotinamid | Last(Universal) Steps | | |
| | Aspartate | | phospho- | | adenylyltransfe-rase; | 5′-Nucleoti- | (outer | Nicotin- | phospo- | e-phospho- | NaMN | | |
| | oxidase | | ribosyl | | nmn co-transporter | dase | membrane | amide | ribosyl | ribosyl | adenylyl | NAD | |
| | EC | Quinolinate | transferase | NMN | (with pnuc) | (periplasm) | p4) | deamidase | transferase | transferase | transferase | synthase | NAD kinase |
| | 1.4.3.16 | synthase | EC 2.4.2.19 | transporter | EC 2.7.7.1 | EC 3.1.3.5 | EC 3.1.2.00 | EC 3.5.1.19 | EC 2.4.2.11 | EC 2.4.2.12 | EC 2.7.7.18 | EC 6.3.5.1 | EC 2.7.1.23 |
| Organism | nadB | nadA | nadC | pnuC | nadR | (nadN*) | (hel*) | pncA | pncB | (nadV*) | nadD | nadE | nadF |
| Escherichia | REC02514 | REC00717 | REC04331 | REC00718 | REC04272 | REC00463? | — | REC01725 | REC04693 | − | REC04566 | REC01697 | REC02551 |
| coli | | | | | | | | | | | | | |
| Klebsiella | 0 | + | + | + | + | +? | − | + | + | − | + | + | + |
| pneumoniae | | | | | | | | | | | | | |
| Salmonella | + | + | + | − | + | +? | − | + | + | − | + | + | + |
| enteritidis | | | | | | | | | | | | | |
| Salmonella | + | + | + | + | + | +? | − | + | + | − | + | + | + |
| typhi | | | | | | | | | | | | | |
| Salmonella | + | + | + | + | + | +? | − | + | + | − | + | + | + |
| typhimurium | | | | | | | | | | | | | |
| Yersinia | + | + | + | + | + | − | − | + | + | − | + | + | + |
| pestis | | | | | | | | | | | | | |
| Yersinia | + | + | + | + | + | − | − | + | + | − | + | + | + |
| pseudotuber- | | | | | | | | | | | | | |
| culosis | | | | | | | | | | | | | |
| Actinobacillus | − | − | − | + | + | + | + | − | − | RAB00645 | − | − | + |
| actinomy- | | | | | | | | | | | | | |
| celemcomitans | | | | | | | | | | | | | |
| Haemophilus | − | − | − | + | + | + | + | − | − | + | − | − | + |
| ducreyi | | | | | | | | | | | | | |
| Haemophilus | − | − | − | 0 | + | RH100461 | RH105145 | − | − | − | − | − | + |
| influenzae | | | | | | | | | | | | | |
| Pasteurella | − | − | − | + | + | + | + | − | − | + | − | − | + |
| multocida | | | | | | | | | | | | | |
| Pseudomonas | + | + | + | + | + | − | − | + | + | − | + | + | + |
| aeruginosa | | | | | | | | | | | | | |
| Pseudomonas | + | + | + | + | + | − | − | + | + | − | + | + | + |
| fluorescens | | | | | | | | | | | | | |
| Mycobacterium | + | + | + | − | + | − | − | + | + | − | + | + | + |
| bovis | | | | | | | | | | | | | |
| Mycobacterium | + | + | + | − | + | − | − | + | + | − | + | + | + |
| tuberculosis | | | | | | | | | | | | | |
| Lactococcus | − | − | − | + | + | +? | − | − | + | − | + | + | + |
| lactis str. | | | | | | | | | | | | | |
| IL 1403 | | | | | | | | | | | | | |
| Nostoc | + | + | + | + | + | − | − | + | + | − | + | + | + |
| punctiforme | | | | | | | | | | | | | |

Comments:
*-in brackets gene names suggested for Haemophilus infuenzae. Others are from E. coli and Salmonella
+?-homologs are present in corresponding genomes, but their functional roles may be different

TABLE 1B
Occurrence and domain composition of NadR homologs found in ERGO data base; taxonomy and genome reference data

| | | | | | NadR domains | | | comments; gene |
|---|---|---|---|---|---|---|---|---|
| | | | NadR homologs | | | Nucleotidyl | | (protein) ID in |
| Organism | Taxonomy | Data Source | RID in ERGO | aa | HTH | transferase | P-loop kinase | other data bases |
| Escherichia | Proteobacteria; | University of | REC04272 | 417 | 8-64 | 65-238 | 239-417 | P27278 |
| coli | gamma subdivision; | Wisconsin | | | | | | |
| | Enterobacteriaceae. | | | | | | | |
| Klebsiella | Proteobacteria; | WUSTL | RKP04607 + | 298 + 112 | 1-57 | 58-231 | 232-410 | N-term + C-term |
| pneumoniae | gamma subdivision; | | RKP04608 | | | | | |
| | Enterobacteriaceae | | | | | | | |
| Salmonella | Proteobacteria; | Salmonella.org | RSEN00775 + | 94 + 293 | 1-57 | 58-231 | 232-387 | N-term + C-term |
| enteritidis | gamma subdivision; | | RSEN01637 | | | | | |
| | Enterobacteriaceae | | | | | | | |
| Salmonella | Proteobacteria; | Sanger Centre | RTY03576 | 410 | 1-57 | 58-231 | 232-410 | |
| typhi | gamma subdivision; | | | | | | | |
| | Enterobacreriaceae | | | | | | | |
| Salmonella | Proteobacteria; | WUSTL | RSY00616 | 410 | 1-57 | 58-231 | 232-410 | |
| typhimurium | gamma subdivision; | | | | | | | |
| | Enterobacteriaceae | | | | | | | |
| Yersinia | Proteobacteria; | Sanger Centre | RYP02013 | 423 | 1-57 | 58-231 | 232-423 | |
| pestis | gamma subdivision; | | | | | | | |
| | Enterobacteriaceac | | | | | | | |
| Yersinia | Proteobacteria; | Extracted from | RYS05348 | 244 | 1-57 | 58-231 | 232->* | |
| pseudo- | gamma subdivision; | EMBL | | | | | | |
| tuberculosis | Enterobacteriaceae | | | | | | | |
| Actinobacillus | Proteobacteria; | Univ. Oklahoma | RAB01716 | 423 | 1-56 | 58-231 | 232-423 | |
| actinomyce- | gamma subdivision; | | | | | | | |
| temcomitans | Pasteurellaceae | | | | | | | |
| Haemophilus | Proteobacteria; | U. Wash. HTSC | RDU01445 + | 308 + 95 | 1-56 | 58-231 | 232-423 | N-term + C-term |
| ducreyi | gamma subdivision; | | RDU01446 | | | | | |
| | Pasteurcellaccae | | | | | | | |
| Haemophilus | Proteobacteria; | TIGR | RH120258 | 407 | − | 38-212 | 213-407 | H10763 P44308 |
| influenzae | gamma subdivision; | | | | | | | |
| | Pasteurellaccae | | | | | | | |
| Pasteurella | Proteobacteria; | U. of Minnesota | RVK00160 | 428 | 1-56 | 58-231 | 232-428 | |
| multocida | gamma subdivision; | Microbial Genome | | | | | | |
| | Pasteurellaceae | Project | | | | | | |
| Moraxella | Proteobacteria; | no genomic data | tr/P71501 | 337 | — | 9-167 | 168-337 | P71501 |
| catarrhalis | gamma subdivision; | | | | | | | |
| | Moraxellaceae; | | | | | | | |
| Pseudomonas | Proteobacteria; | University of | RPA02808 | 175 | — | — | 1-175 | PA1957 |
| aeruginosa | gamma subdivision; | Washington | | | | | | |
| | Pseudomonas group | | | | | | | |
| Pseudomonas | Proteobacteria; | DOE JGI | RPU05722 | 183 | — | — | 11-183 | |
| fluorescens | gamma subdivision; | | | | | | | |
| | Pseudomonas group | | | | | | | |
| Mycobacterium | Finnicutes; | Sanger Centre | RMB03209 | 319 | — | 1-152 | 253-319 | RV0212C P96394 |
| bovis | Actinobacteria; | | | | | | | |
| | Actinobacteridae; | | | | | | | |
| | Actinomycetales; | | | | | | | |
| | Corynebacterineae; | | | | | | | |
| | Mycobacteriaceae | | | | | | | |
| Mycobacterium | Firmicures; | TIGR | RMT01595 | 323 | — | 1-148 | 149-323 | |
| tuberculosis | Actinobacteria; | | | | | | | |
| | Actinobacteridae; | | | | | | | |
| | Actinomycetales; | | | | | | | |
| | Corynebacterineae; | | | | | | | |
| | Mycobacteriaceae | | | | | | | |
| Lactococcus | Firmicutes; Bacillus/ | INRA | RI.1.X02029 | 379 | — | 18-180 | 181-379 | |
| lactis str. | Clostridium group; | | | | | | | |
| 1L 1403 | Streptococcaceae | | | | | | | |
| Nostoc | Cyanobacteria; | DOE JGI | RNPU01914 | 342 | — | 7-155 | 156-342 | |
| punctiforme | Nostocales; | | | | | | | |
| | Nostocaceae | | | | | | | |

Comments: Amino acid sequences of NadR homologs of Klebsiella pneumoniae (RKP04607 and RKP04608), Salmonella enteritidis (RSEN00775 and RSEN01637), and Haemophilus ducreyi (RDU01445 and RDU01446) appear as two ORFs due to frame shifts or broken contigs.
Closest homologs were used for numeration of the domain boundaries
*-in Yersinia pseudotuberculosis the contig is broken; and there is not enough genomic sequences to claim presence or absence of the C-terminal part Homologs of pnuC identified in this set of genomes are listed in the last two columns
Two nadR proteins characterized in this study are shown in bold

TABLE 2

| | NMNATse | NmR Kinase | |
|---|---|---|---|
| | Un/mg | Un/mg | RATIO |
| NadR Salmonella | 0.04 | 5 | 125 |
| NadR H. influenzae | 0.9 | 0.02 | 0.02 |
| NadR H. influenzae; | 3 | <0.003 | <0.001 |
| N-terminal domain | | | |

TABLE 3

Primers used for PCR amplification and cloning

| SEQ | | orient, in | Site for | | | Designation in this |
|---|---|---|---|---|---|---|
| ID | primer with site | the gene | cloning | Changes | Genome | study |
| 43 | gggccATCgCAAAAACAAAAGAGAAAA | 5′−>3′ | NcoI | S39A | Haemophilus | NadR_HI_Ndomain |
| | AAGTCGGTGTCATTTTC | | | | influenzae | |
| 44 | ggggtcgactcaAAAGAAAGGACGAGC | 3′−>5′ | SalI | Add TGA | | |
| | TTCTTTCGGAATAAACTTC | | | | | |
| 43 | gggccATCgCAAAAACAAAAGAGAAAA | 5′−>3′ | NcoI | S39A) | Haemophilus | NadR_HI_Thuncated |
| | AAGTCGGTGTCATTTTC | | | | influenzae | |
| 45 | gggtcgacTCATTGAGATGTCCCTTT | 3′−>5′ | SalI | none | | |
| | TATAGGAAAGGTTGTG | | | | | |
| 46 | gggtcaTGaCATCGTTCGACTATCTCA | 5′−>3′ | BspH1 | GTG- | Salmonella | NadR_SY |
| | AAACCGCG | | | >aTG;TCA- | typhimurium | |
| | | | | >aCA | | |
| 47 | ggggtcgacTTAactcgagCCCTGCTC | 3′−>5′ | SalI | XhoI,STOP,SalI | | |
| | GCCCATCATCTCTTTC | | | (with additional | | |
| | | | | SS) | | |
| 48 | gggccATGGAAAATTCCGAGAAGACTG | 5′−>3′ | NcoI | | Homo sapiens | NadD2_{HS} |
| | AAGTGGTTCTCCTTGC | | | | | |
| 49 | ggggtcgacCTATGTCTTAGCTTCTGC | 3′−>5′ | SalI | | | |
| | AGTGTTTCTCTGCAAAGGGG | | | | | |

What is claimed is:

1. An isolated nucleic acid molecule comprising an isolated polynucleotide having a nucleotide sequence at least 95% identical to a sequence selected from the group consisting of:
(a) a nucleotide sequence encoding a biologically active polypeptide fragment of a nicotinamide ribonucleoside kinase (NRKse) domain of SEQ ID NO: 2, 4,6, 8, 10, 12, 14, 16, 18, 20, 22, 24, 26, 28, 30, 32, 34, 36, 38, 40, or 42;
(b) a nucleotide sequence encoding an NRKse domain of SEQ ID NO: 2, 4,6, 8, 10, 12, 14, 16, 18, 20, 22, 24, 26, 28, 30, 32, 34, 36, 38, 40, or 42; and
(c) a nucleotide sequence capable of hybridizing under stringent conditions to any one of the polynucleotides specified in (a) or (b).

2. The isolated nucleic acid molecule of claim 1, wherein the polynucleotide comprises a nucleotide sequence encoding a nicotinamide ribonucleoside kinase (NRKse).

3. The isolated nucleic acid molecule of claim 1, wherein the polynucleotide comprises a nucleotide sequence encoding the NRKse domain of SEQ ID NO: 2, 4, 6, 8, 10, 12, 14, 16, 18, 20, 22, 24, 26, 28, 30. 32, 34, 36, 38, 40, or 42.

4. The isolated nucleic acid molecule of claim 1, wherein the polynucleotide comprises the entire nucleotide sequence of a NRKse domain of SEQ ID NO: 1, 3, 5, 7, 9, 11, 13, 15, 17, 19, 21, 23, 25, 27, 29, 31, 33, 35, 37, 39, or 41.

5. The isolated nucleic acid molecule of claim 2, wherein the nucleotide sequence comprises sequential nucleotide deletions from either terminus of the nucleotide sequence encoding said NRKse.

6. The isolated nucleic acid molecule of claim 3, wherein the nucleotide sequence comprises sequential nucleotide deletions from either terminus of the nucleotide sequence encoding said NRKse.

7. A recombinant vector comprising the isolated nucleic acid molecule of claim 1.

8. A method of making a recombinant host cell comprising the isolated nucleic acid molecule of claim 1, the method comprising introducing into a host cell the isolated nucleic acid molecule of claim 1.

9. A recombinant host cell produced by the method of claim 8.

10. The recombinant host cell of claim 9 comprising a vector.

11. An isolated polypeptide comprising an isolated amino acid sequence at least 95% identical to a sequence selected from the group consisting of:
(a) a biologically active polypeptide fragment of an NRKse domain of SEQ ID NO: 2, 4, 6, 8, 10, 12, 14, 16, 18, 20, 22, 24, 26, 28, 30, 32, 34, 36, 38, 40, or 42; and
(b) a polypeptide comprising the sequence of an NRKse domain of SEQ ID NO: 2, 4, 6, 8, 10, 12, 14, 16, 18, 20, 22, 24, 26, 28, 30, 32, 34, 36, 38, 40, or 42.

12. The isolated polypeptide of claim 11, wherein the polypeptide comprises sequential amino acid deletions from either the C-terminus or the N-terminus of NRKse.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The isolated polypeptide produced by the method of claim 15.

17. A method for determining whether a compound is a modulator of NRKse, the method comprising:
(a) contacting the isolated polypeptide of claim 11 with said compound; and
(b) detecting an alteration in the activity of the polypeptide in the presence of the compound as an indication that said compound is a modulator of NRKse.

18. A method for modulating NRKse activity in an organism, the method comprising contacting the organism with a modulator identified by the method of claim 17, thereby modulating NRKse activity in the organism.

19. The method of claim 18, wherein the organism is a pathogen and modulation comprises decreasing NRKse activity in the organism, thereby inhibiting the pathogenicity of said organism

20. The method of claim 18, wherein the modulation comprises increasing NRKse activity in the organism.

21. The method of claim 17, further comprising:
(c) contacting said compound with nicotinamide mononucleotide adenylyl transferase (NMNATse); and
(d) detecting an alteration in the activity of said NMNATse in the presence of said compound as an indication that said compound is a modulator of NMNATse.

22. A method for producing β-nicotinamide mononucleotide (NMN) or an analog thereof, the method comprising:
contacting 5-nicotinamide ribonucleoside (NMR) or an analog thereof with the biologically active polypeptide of claim 11, thereby producing NMN or an analog thereof.

23. The method of claim 22, wherein the analog is 3′deazaguanosine or tiazofurin.

24. The method of claim 22, further comprising contacting NMN or the analog thereof with nicotinamide mononucleotide adenylyl transferase (NMNATse), thereby producing β-nicotinamide adenine dinucleotide (NAD) or a dinucleotide of said analog.

25. The method of claim 24, wherein the biologically active polypeptide and NMNATse are provided as a fusion protein.
